(19) United States
(12) Patent Application Publication
(10) Pub. No.: US 2003/0153007 A1
(43) Pub. Date: Aug. 14, 2003

(54) Automated systems and methods for analysis of protein post-translational modification

(76) Inventors: Jian Chen, Mississauga (CA); Joseph Michel Daniel Figeys, Pickering (CA); Brett Larsen, Toronto (CA); Forest M. White, Charlottesville, VA (US)

Correspondence Address: ROPES & GRAY LLP, ONE INTERNATIONAL PLACE, BOSTON, MA, 02110-2624, US

(21) Appl. No.: 10/330,861

(22) Filed: Dec. 26, 2002

Related U.S. Application Data
(60) Provisional application No. 60/343,645, filed on Dec. 28, 2001.
(60) Provisional application No. 60/361,236, filed on Mar. 1, 2002.

(51) Int. Cl. G01N 33/53; B01D 59/44; H01J 49/00

(52) U.S. Cl. 435/7.1; 250/282

(57) ABSTRACT

Methods and systems of applying mass spectrometry to the analysis of peptides and amino acids, especially in the proteome setting. More particularly, the invention relates to a mass spectrometry-based method for detection of amino acid modifications, such as phosphorylation.

### REFERENCE TO RELATED APPLICATIONS

The present application claims priority to U.S. Provisional application No. 60/343,645, filed on Dec. 28, 2001, and U.S. Provisional application No. 60/361,236, filed on Mar. 1, 2002, the entire contents of which are all incorporated by reference herein.

### FIELD OF THE INVENTION

This invention is in the field of proteomics, and applies mass spectrometry to the analysis of peptides and amino acids. More particularly, the invention relates to a mass spectrometry-based method for detection of amino acid modifications, such as phosphorylation.

### BACKGROUND TO THE INVENTION

With the availability of a burgeoning sequence database, genomic applications demand faster and more efficient methods for the global screening of protein expression in cells. However, the complexity of the cellular proteome expands substantially if protein post-translational modifications are also taken into account.

Dynamic post-translational modification of proteins is important for maintaining and regulating protein structure and function. Among the several hundred different types of post-translational modifications characterized to date, protein phosphorylation plays a prominent role. Enzyme-catalyzed phosphorylation and dephosphorylation of proteins is a key regulatory event in the living cell. Complex biological processes such as cell cycle, cell growth, cell differentiation, and metabolism are orchestrated and tightly controlled by reversible phosphorylation events that modulate protein activity, stability, interaction and localization. Perturbations in phosphorylation states of proteins, e.g. by mutations that generate constitutively active or inactive protein kinases and phosphatases, play a prominent role in oncogenesis. Comprehensive analysis and identification of phosphoproteins combined with exact localization of phosphorylation sites in those proteins (‘phosphoproteomics’) is a prerequisite for understanding complex biological systems and the molecular features leading to disease.

It is estimated that ⅓ of all proteins present in a mammalian cell are phosphorylated and that kinases, enzymes responsible for that phosphorylation, constitute about 1-3% of the expressed genome. Organisms use reversible phosphorylation of proteins to control many cellular processes including signal transduction, gene expression, the cell cycle, cytoskeletal regulation and apoptosis. A phosphate group can modify serine, threonine, tyrosine, histidine, arginine, lysine, cysteine, glutamic acid and aspartic acid residues. However, the phosphorylation of hydroxyl groups at serine (90%), threonine (10%), or tyrosine (0.05%) residues are the most prevalent, and are involved among other processes in metabolism, cell division, cell growth, and cell differentiation. Because of the central role of phosphorylation in the regulation of life, much effort has been focused on the development of methods for characterizing protein phosphorylation.

The identification of phosphorylation sites on a protein is complicated by the facts that proteins are often only partially phosphorylated and that they are often present only at very low levels. Therefore techniques for identifying phosphorylation sites should preferably work in the low picomole to sub-picomole range.

Traditional methods for analyzing O-phosphorylation sites involve incorporation of ³²P into cellular proteins via treatment with radiolabeled ATP. The radioactive proteins can be detected during subsequent fractionation procedures (e.g. two-dimensional gel electrophoresis or high-performance liquid chromatography [HPLC]). Proteins thus identified can be subjected to complete hydrolysis and the phosphoamino acid content determined. The site(s) of phosphorylation can be determined by proteolytic digestion of the radiolabeled protein, separation and detection of phosphorylated peptides (e.g. by two-dimensional peptide mapping), followed by peptide sequencing by Edman degradation. These techniques can be tedious, require significant quantities of the phosphorylated protein and involve the use of considerable amounts of radioactivity.

In recent years, mass spectrometry (MS) has become an increasingly viable alternative to more traditional methods of phosphorylation analysis. The most widely used method for selectively enriching phosphopeptides from mixtures is immobilized metal affinity chromatography (IMAC). In this technique, metal ions, usually Fe3+ or Ga3+, are bound to a chelating support. Phosphopeptides are selectively bound because of the affinity of the metal ions for the phosphate moiety. The phosphopeptides can be released using high pH or phosphate buffer, the latter usually requiring a further desalting step before MS analysis. Limitations of this approach include possible loss of phosphopeptides because of their inability to bind to the IMAC column, difficulty in the elution of some multiply phosphorylated peptides, and background from unphosphorylated peptides (typically acidic in nature) that have affinity for immobilized metal ions. Two types of chelating resin are commercially available, one using iminodiacetic acid and the other using nitrilotriacetic acid. Some groups have observed that iminodiacetic acid resin is less specific than nitrilotriacetic acid, whereas another study reported little difference between the two. Several studies have examined off-line MS analysis of IMAC-separated peptides.

Recently, two groups have described protocols to achieve this goal. Oda et al. (Nat Biotechnol. 2001 19:379-82) start with a protein mixture in which cysteine reactivity is removed by oxidation with performic acid. Base hydrolysis is used to induce -elimination of phosphate from phosphoserine and phosphothreonine, followed by addition of ethanedithiol to the alkene. The resulting free sulfhydryls are coupled to biotin, allowing purification of phosphoproteins by avidin affinity chromatography. Following elution of phosphoproteins and proteolysis, enrichment of phosphopeptides is carried out by a second round of avidin purification. Disadvantages of this approach include the failure to detect phosphotyrosine containing peptides and generation of diastereoisomers in the derivatization step.

The approach suggested by the Zhou et al. (Nat Biotechnol 2001 19:375-378) circumvents these problems but involves a six step derivatization/purification protocol for tryptic peptides that requires more than 13 hrs to complete and affords only a 20% yield from picomoles of phosphopeptide starting material. The method begins with a proteolytic digest that has been reduced and alkylated to eliminate reactivity from cysteine residues. Following N-terminal and C-terminal protection, phosphoramidate adducts at phosphorylated residues are formed by carbodiimide condensation with cystamine. The free sulfhydryl groups produced from this step are covalently captured onto glass beads coupled to iodoacetic acid. Elution with trifluoroacetic acid then regenerates phosphopeptides for analysis by mass spectrometry.

### SUMMARY OF THE INVENTION

One aspect of the present provides a method for identifying modified amino acids within a protein by combining affinity purification and mass spectroscopy in a manner which is amenable to high throughput and automation. In general, the subject method makes use of affinity capture reagents for isolating, from a protein sample, those proteins which have been post-translationally modified with a moiety of interest. In order to improve the selectivity/efficiency of the affinity purification step, the protein samples to be analyzed are chemically modify at least one of the C-terminal carboxyl, the N-terminal amine and amino acid side chains of the proteins which may interfere with the selectively of the affinity purification step for the post-translational modification of interest. Proteins which are isolated based on post-translational modifications are than analyzed by mass spectroscopy in order to identify patterns of modification across a proteome, and/or to provide the identity of proteins in the sample which are modified or shows changes in modification status between two different samples.

In certain preferred embodiments, the proteins are cleaved into smaller peptide fragments before, after or during the chemical modification step. For instance, the proteins can be fragmented by enzymatic hydrolysis to produce peptide fragments having carboxy-terminal lysine or arginine residues. In certain preferred embodiments, the proteins are fragmented by treatment with trypsin.

In certain embodiments, the proteins are mass-modified with isotopic labels before, after or during the chemical modification step.

In certain embodiments, the proteins are further separated by reverse phase chromatography before analysis by mass spectroscopy.

There are a variety of mass spectroscopy techniques which can be employed in the subject method. In certain preferred embodiments, the isolated proteins are identified from analysis using tandem mass spectroscopy techniques, such as LC/MS/MS. Where the proteins have been further fragmented with trypsin or other predictable enzymes, the molecular weight of a fragment as determined from the mass spectroscopy data can be used to identify possible matches in molecular weight databases indexed by predicted molecular weights of protein fragments which would result under similar conditions as the fragments generated in the subject method. However, the subject method can be carried out using mass spectroscopy techniques which produce amino acid sequence mass spectra for the isolated proteins or peptide fragments. The sequence data can be used to search one or more sequence databases.

In certain preferred embodiments, the method is used to identify phosphorylated proteins or changes in the phosphorylation pattern amongst a group of proteins. In such embodiments, the affinity capture reagent can be an immobilized metal affinity chromatography medium, and the step of processing the protein samples includes chemically modifying the side chains of glutamic acid and aspartic acid residues to neutral derivatives, such as by alkyl-esterification.

The subject method is amenable to analysis of multiple different protein samples, particularly in a multiplex fashion. In such embodiments, the proteins or fragments thereof are isotopically labeled in a manner which permits discrimination of mass spectroscopy data between protein samples. That is, a mass spectra on the mixture of various protein samples can be deconvoluted to determine the sample origin of each signal observed in the spectra. In certain embodiments, this technique can be used to quantitated differences in phosphorylation (or other modification) levels between samples prepared under different conditions and admixed prior to MS analysis.

In certain embodiments, the subject method is used for analyzing a phosphoproteome. For example, the proteins in the sample can be chemically modify at glutamic acid and aspaitic acid residues, such as by alkyl-esterification, to generate neutral side chains at those positions. The phosphorylated proteins in the same are then isolated by immobilized metal affinity chromatography, and analyzed by mass spectroscopy. In preferred embodiments, the proteins are cleaved, e.g., by trypsin digestion or the like, into smaller peptide fragments before, after or during the step of chemically modify the glutamic acid and aspartic acid residues. In one embodiment, the subject method is carried out on multiple different protein samples, and proteins which a differentially phosphorylated between two or more protein samples are identified. That data can, for instance, be used to generate or augment databases with the identity of proteins which are determined to be phosphorylated.

Another aspect of the invention provides a method for identifying a treatment that modulates a modification of amino acid in a target polypeptide. In general, this method is carried out by providing a protein sample which has been subjected to a treatment of interest, such as treatment with ectopic agents (drugs, growth factors, etc). The protein samples can also be derived from normal cells in different states of differentiation or tissue fate, or derived from normal and diseased cells. Following the affinity purification/MS method set forth above, the identity of proteins which are differentially modified in the treated protein sample relative to an untreated sample or control sample can determined. From this identification step, one can determine whether the treatment results in a pattern of changes in protein modification, relative to the untreated sample or control sample, which meet a pre-selected criteria. Thus, one can use this method to identify compounds likely to mimic the effect of a growth factor by scoring for similarities in phosphorylation patterns when comparing proteins from the compound-treated cells with proteins from the growth factor treated cells. The treatment of interest can include contacting the cell with such compounds as growth factors, cytokines, hormones, or small chemical molecules. In certain embodiments, the method is carried out with various members of a chemically diverse library.

Another aspect of the invention provides a diagnostic method. In general, using any one of the suitable methods of the instant invention, one can generate profiles of phosphopeptides of related biological samples (for example, disease tissue vs. normal tissue, or stem/progenitor cells vs. differentiated cells, cells treated by certain agents (such as pharmaceutical drugs or drug candidates) vs. those untreated control, or cells at different developmental stages, etc.), and compare these profiles of phosphopeptides. If a statistically significant difference in the profile is present between the samples being compared, a conclusion can be made about the status of these samples. In this regard, the instant invention can be used to diagnose the presence of a certain disease state, using a biopsy obtained from a patient. The instant invention can also be used to sort biological samples into different categories based on the similarity of their respective profiles.

It should be understood that profiles of amino acid modifications other than phosphorylation can also be obtained using the subject method, and thus such methods also fall within the scope of the invention.

Yet another aspect of the present invention provides a method of conducting a drug discovery business. Using the assay described above, one determines the identity of a compound that produces a pattern of changes in protein modification, relative to the untreated sample or control sample, which meet a preselected criteria. Therapeutic profiling of the compound identified by the assay, or further analogs thereof, can be carried out for determining efficacy and toxicity in animals. Compounds identified as having an acceptable therapeutic profile can then be formulated as part of a pharmaceutical preparation. In certain embodiments, the method can include the additional step of establishing a distribution system for distributing the pharmaceutical preparation for sale, and may optionally include establishing a sales group for marketing the pharmaceutical preparation. In other embodiments, rather than carry out the profiling and/or formulation steps, one can license, to a third party, the rights for further drug development of compounds that are discovered by the subject assay to alter the level of modification of the target polypeptide.

Yet another aspect of the present invention provides a method of conducting a drug discovery business in which, after determining the identity of a protein that is post-translationally modified under the conditions of interest, the identity of one or more enzymes which catalyze the post-translational modification of the identified protein under the conditions of interest is determined. Those enzyme(s) are then used as targets in drug screening assays for identifying compounds which inhibit or potentiate the enzymes and which, therefore, can modulate the post-translational modification of the identified protein under the conditions of interest.

### REFERENCE TO THE DRAWINGS

FIG. 1. Five nonphosphorylated proteins; glyceraldehyde 3-phosphate dehydrogenase, bovine serum albumin, carbonic anhydrase, ubiquitin, and β-lactoglobulin (Sigma Chemical Co., St. Louis, Mo.) (100 nmol each) in 1.1 ml of 100 mM ammonium bicarbonate (pH 8) were digested with trypsin (20 μg) (Promega, Madison, Wis.) for 24 h at 37° C. The reaction was quenched with 65 μl of glacial acetic acid, and the mixture was then diluted to final volume of 50 ml with 0.1% acetic acid. To this solution was added 500 pmol of HPLC purified phosphopeptide, DRVpYIHPF (SEQ ID No: 1) (Novabiochem, San Diego, Calif.), in 0.1% acetic acid (2 μL of a 250 pmol/μL stock solution). An aliquot of the standard mixture (100 μl) was lyophilized and redissolved in 100 μl of 2 N methanolic HCl. This latter solution was prepared by dropwise addition of 160 μl of acetyl chloride with stirring to 1 ml of methanol. Esterification was allowed to proceed for 2 h at room temperature. Solvent was removed by lyophilization and the resulting sample was redissolved in 100 μl of solution containing equal volumes of methanol, water and acetonitrile. Phosphate methyl esters are not observed under these conditions. Mass spectra recorded by a combination of immobilized metal affinity chromatography (IMAC) and nano-flow HPLC microelectrospray ionization mass spectrometry on the phosphopeptide, DRVpYIHPF (SEQ ID No: 1), present at the level of 10 fmol/μl in a mixture containing tryptic peptides from 5 proteins at the level of 2 pmol/μl. Aliquots corresponding to 0.5 μl of the above solutions (tryptic peptides from 1 pmol of each protein plus 5 fmol of phosphopeptide, DRVpYIHPF, SEQ ID No: 1) were analyzed by mass spectrometry. (A) Selected ion chromatogram, SIC, or plot of the ion current vs scan number for m/z 564.5 corresponding to the (M+2H)⁺⁺ of the phosphopeptide, DRVpYIHPF (SEQ ID No: 1). (B) MS/MS spectrum characteristic of the sequence, DRVpYIHPF (SEQ ID No: 1), recorded on ions of m/z 564.5 in scans 610-616. (C) Electrospray ionization mass spectrum recorded during this same time interval. Abundant ions from tryptic peptides non-specifically bound to the IMAC column obscure the signal at m/z 564.5 for DRVpYIHPF (SEQ ID No: 1). (D) SIC for m/z 578.5 corresponding to the (M+2H)++ ion for the dimethyl ester of DRVpYIPF (SEQ ID No: 1). (E) MS/MS spectrum characteristic of the sequence, DRVpYIPF (SEQ ID No: 1), recorded in on ions of m/z 578.5 in scans 151-163. (F) Electrospray ionization mass spectrum recorded in scan 154 showing the parent ion, m/z 578.5 for the phosphopeptide dimethyl ester and the absence of signals for tryptic peptides non specifically bound to the IMAC column.

FIG. 2. The phosphopeptide B-casein was analyzed using the subject invention. Top: extracted ion chromatograph from the HPLC separation showing the B-casein peak at 85.33 min. Bottom: Casein MS/MS scan at m/z=1031.6, showing individual peptide fragments of the phosphopeptide components of Casein.

FIG. 3. Schematic of exemplary system for automating the subject method.

### DETAILED DESCRIPTION OF THE INVENTION

Definitions

For convenience, certain terms employed in the specification, examples, and appended claims are collected here.

“Affinity capture reagent” as used herein means reagents that has affinity for proteins, including their backbone and side-chains, either modified or naturally occurring, due to, for example, electrostatic, hydrophobic, ionic and/or hydrogen-bond interactions under physiological/experimental conditions. An exemplary affinity capture reagent is resin used in IMAC.

“Binding,” “bind” or “bound” refers to an association, which may be a stable association between two molecules, e.g., between a modified protein ligand an affinity capture reagent, due to, for example, electrostatic, hydrophobic, ionic and/or hydrogen-bond interactions under physiological conditions.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A “chimeric protein” or “fusion protein” is a fusion of a first amino acid sequence encoding a polypeptide with a second amino acid sequence defining a domain foreign to and not substantially homologous with any domain of the protein. A chimeric protein may present a foreign domain which is found (albeit in a different protein) in an organism which also expresses the first protein, or it may be an “interspecies”, “intergenic”, etc. fusion of protein structures expressed by different kinds of organisms.

The terms “compound”, “test compound” and “molecule” are used herein interchangeably and are meant to include, but are not limited to, peptides, nucleic acids, carbohydrates, small organic molecules, natural product extract libraries, and any other molecules (including, but not limited to, chemicals, metals and organometallic compounds).

The phrases “conserved residue” “or conservative amino acid substitution” refer to grouping of amino acids on the basis of certain common properties. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer., Principles of Protein Structure, Springer-Verlag). Examples of amino acid groups defined in this manner include:

(i) a charged group, consisting of Glu and Asp, Lys, Arg and His,

(ii) a positively-charged group, consisting of Lys, Arg and His,

(iii) a negatively-charged group, consisting of Glu and Asp,

(iv) an aromatic group, consisting of Phe, Tyr and Trp,

(v) a nitrogen ring group, consisting of His and Trp,

(vi) a large aliphatic nonpolar group, consisting of Val, Leu and Ile,

(vii) a slightly-polar group, consisting of Met and Cys,

(viii) a small-residue group, consisting of Ser, Thr, Asp, Asn, Gly, Ala, Glu, Gln and Pro,

(ix) an aliphatic group consisting of Val, Leu, Ile, Met and Cys, and

(x) a small hydroxyl group consisting of Ser and Thr.

In addition to the groups presented above, each amino acid residue may form its own group, and the group formed by an individual amino acid may be referred to simply by the one and/or three letter abbreviation for that amino acid commonly used in the art.

The term “DNA sequence encoding a polypeptide” may refer to one or more genes within a particular individual. As is well known in the art, genes for a particular polypeptide may exist in single or multiple copies within the genome of an individual. Such duplicate genes may be identical or may have certain modifications, including nucleotide substitutions, additions or deletions, which all still code for polypeptides having substantially the same activity. Moreover, certain differences in nucleotide sequences may exist between individual organisms, which are called alleles. Such allelic differences may or may not result in differences in amino acid sequence of the encoded polypeptide yet still encode a protein with the same biological activity.

The term “domain” as used herein refers to a region within a protein that comprises a particular structure or function different from that of other sections of the molecule.

“Exogenous” means caused by factors or an agent from outside the organism or system, or introduced from outside the organism or system, specifically: not normally synthesized within the organism or system. A fusion/tagged protein expressed from an introduced plasmid may be considered exogenous to the host cell expressing the fusion protein, although the host itself may express an endogenous version of the same protein.

“Extracellular factor” includes a molecule or a change in the environment that is transduced intracellularly via cell surface proteins (e.g. cell surface receptors) that interact, directly or indirectly, with a signal. An extracellular factor includes any compound or substance that in some manner specifically alters the activity of a cell surface protein. Examples of such signals or factors include, but are not limited to growth factors, that bind to cell surfaces and/or intracellular receptors and ion channels and modulate the activity of such receptors and channels. The signals and factors include analogs, derivatives, mutants, and modulators of such growth factors.

“Intracellular factor” includes a molecule or a change in the cell environment that is transduced in the cell via cytoplasmic proteins that interact, directly or indirectly with a signal. An intracellular factor includes any compound or substance that in some manner specifically alters the activity of a cytoplasmic protein involved in a biological or signal transduction pathway.

“High throughput” refers to the ability to process large amount of samples in a given process, method, or assay, etc. In a preferred embodiment, the high throughput process is conducted with an automated machine(s), which is optionally controlled by computer software or human or both.

“Homology” or “identity” or “similarity” refers to sequence similarity between two peptides or between two nucleic acid molecules, with identity being a more strict comparison. Homology and identity can each be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences. An “unrelated” or “non-homologous” sequence shares less than 40 % identity, though preferably less than 25% identity, with one of the--sequences of the present invention.

As used herein, the term “gene” or “recombinant gene” refers to a nucleic acid comprising an open reading frame encoding a polypeptide of the present invention, including both exon and (optionally) intron sequences. A “recombinant gene” refers to nucleic acid encoding a polypeptide and comprising exon coding sequences, though it may optionally include intron sequences derived from a chromosomal gene. The term “intron” refers to a DNA sequence present in a given gene which is not translated into protein and is generally found between exons.

The term “GI” or “GI Number” or “GI No.” refers to database access number (such as gene bank) for genes and/or proteins useful for retrieving sequence and other related information.

“Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “nonhomologous” shares less than 20% identity, though preferably less than 15% identity with a sequence of the present invention. Similarly, “homology” or “homologous” refers to sequences that are at least 20%, 25%, 30%, 35%, 40%, 45%, 50%, 55%, 60%, 65%, 70%, 75%, 80%, 85%, 90%, or even 95% to 99% identical to one another.

The term “homology” describes a mathematically based comparison of sequence similarities which is used to identify genes or proteins with similar functions or motifs. The nucleic acid and protein sequences of the present invention may be used as a “query sequence” to perform a search against public databases to, for example, identify other family members, related sequences or homologs. Such searches can be performed using the NBLAST and XBLAST programs (version 2.0) of Altschul, et al. (1990) J Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and-BLAST) can be used.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both polypeptide and DNA databases.

Databases with individual sequences are described in Methods in Enzymology, ed. Doolittle, supra. Some exemplary public databases include GenBank, EMBL, DNA Database of Japan (DDBJ), SwissProt, PIR and other databases derived therefrom. In comparing a new nucleic acid with known sequences, several alignment tools are available. Examples include PileUp, which creates a multiple sequence alignment, and is described in Feng et al., J. Mol. Evol. (1987) 25:351-360. Another method, GAP, uses the alignment method of Needleman et al., J. Mol. Biol. (1970) 48:443-453. GAP is best suited for global alignment of sequences. A third method, BestFit, functions by inserting gaps to maximize the number of matches using the local homology algorithm of Smith and Waterman, Adv. Appl. Math. (1981) 2:482-489. Alternatively, certain commercial software packages such as LaserGene from DNAStar inc. can be used for certain aspects of sequence analysis. Multiple software and databases may be used in any analysis.

The term “Interacting Protein” is meant to include polypeptides that interact either directly or indirectly with another protein. Direct interaction means that the proteins may be isolated by virtue of their ability to bind to each other (e.g. by coimmunoprecipitation or other means). Indirect interaction refers to proteins which require another molecule in order to bind to each other. Alternatively, indirect interaction may refer to proteins which never directly bind to one another, but interact via an intermediary.

The term “isolated”, as used herein with reference to the subject proteins and protein complexes, refers to a preparation of protein or protein complex that is essentially free from contaminating proteins that normally would be present in association with the protein or complex, e.g., in the cellular milieu in which the protein or complex is found endogenously. Thus, an isolated protein complex is isolated from cellular components that normally would “contaminate” or interfere with the study of the complex in isolation, for instance while screening for modulators thereof. It is to be understood, however, that such an “isolated” complex may incorporate other proteins the modulation of which, by the subject protein or protein complex, is being investigated.

Polypeptides referred to herein as “mammalian homologs” of a protein refers to other mammalian paralogs, or other mammalian orthologs. “Analyzing a protein by mass spectrometry” or similar wording refers to using mass spectrometry to generate information which may be used to identify or aid in identifying a protein. Such information includes, for example, the mass or molecular weight of a protein, the amino acid sequence of a protein or protein fragment, a peptide map of a protein, and the purity or quantity of a protein.

The term “motif” as used herein refers to an amino acid sequence that is commonly found in a protein of a particular structure or function. Typically a consensus sequence is defined to represent a particular motif. The consensus sequence need not be strictly defined and may contain positions of variability, degeneracy, variability of length, etc. The consensus sequence may be used to search a database to identify other proteins that may have a similar structure or function due to the presence of the motif in its amino acid sequence. For example, on-line databases such as GenBank or SwissProt can be searched with a consensus sequence in order to identify other proteins containing a particular motif. Various search algorithms and/or programs may be used, including FASTA, BLAST or ENTREZ. FASTA and BLAST are available as a part of the GCG sequence analysis package (University of Wisconsin, Madison, Wis.). ENTREZ is available through the National Center for Biotechnology Information, National Library of Medicine, National Institutes of Health, Bethesda, Md.

“Proteome” refers to all the proteins that can be encoded by a given genome, which is in turn all the genetic material (including all the genes) of a given organism. Not all proteins within a given proteome are necessarily expressed at the same time, in the same cell type/tissue origin. Due to changes in conditions such as developmental, environmental, physiological, or pathological conditions, any given tissue/cell type may only express a fraction of the total number of proteins that can be encoded by a given genome (or, a fraction of the total proteome). “Proteome” may also refer to the entire complement of proteins expressed by a given tissue or cell type.

The term “purified protein” refers to a preparation of a protein or proteins which are preferably isolated from, or otherwise substantially free of, other proteins normally associated with the protein(s) in a cell or cell lysate. The term “substantially free of other cellular proteins” (also referred to herein as “substantially free of other contaminating proteins”) is defined as encompassing individual preparations of each of the component proteins comprising less than 20% (by dry weight) contaminating protein, and preferably comprises less than 5% contaminating protein. Functional forms of each of the component proteins can be prepared as purified preparations by using a cloned gene as described in the attached examples. By “purified”, it is meant, when referring to component protein preparations used to generate a reconstituted protein mixture, that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” as used herein preferably means at least 80% by dry weight, more preferably in the range of 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above. “Isolated” and “purified” do not encompass either protein in its native state (e.g. as a part of a cell), or as part of a cell lysate, or that have been separated into components (e.g., in an acrylamide gel) but not obtained either as pure (e.g. lacking contaminating proteins) substances or solutions. The term isolated as used herein also refers to a component protein that is substantially free of cellular material or culture medium when produced by recombinant DNA techniques, or chemical precursors or other chemicals when chemically synthesized.

The term “recombinant protein” refers to a protein of the present invention which is produced by recombinant DNA techniques, wherein generally DNA encoding the expressed protein is inserted into a suitable expression vector which is in turn used to transform a host cell to produce the heterologous protein. Moreover, the phrase “derived from”, with respect to a recombinant gene encoding the recombinant protein is meant to include within the meaning of “recombinant protein” those proteins having an amino acid sequence of a native protein, or an amino acid sequence similar thereto which is generated by mutations including substitutions and deletions of a naturally occurring protein.

Genetic techniques, which allow for the expression of transgenes can be regulated via site-specific genetic manipulation in vivo, are known to those skilled in the art. For instance, genetic systems are available which allow for the regulated expression of a recombinase that catalyzes the genetic recombination of a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of one of the subject target gene polypeptides. For example, excision of a target sequence which interferes with the expression of a recombinant target gene, such as one which encodes an antagonistic homolog or an antisense transcript, can be designed to activate expression of that gene. This interference with expression of the polypeptide can result from a variety of mechanisms, such as spatial separation of the target gene from the promoter element or an internal stop codon. Moreover, the transgene can be made wherein the coding sequence of the gene is flanked by recombinase recognition sequences and is initially transfected into cells in a 3′ to 5′ orientation with respect to the promoter element. In such an instance, inversion of the target sequence will reorient the subject gene by placing the 5′ end of the coding sequence in an orientation with respect to the promoter element which allows for promoter driven transcriptional activation.

“Phospho-protein” is meant a polypeptide that can be potentially phosphorylated on at least one residue, which can be either tyrosine or serine or threonine or any combination of the three. Phosphorylation can occur constitutively or be induced.

“Post-translational modification” is meant any changes/modifications that can be made to the native polypeptide sequence after its initial translation. It includes, but are not limited to, phosphorylation/dephosphorylation, prenylation, myristoylation, palmitoylation, limited digestion, irreversible conformation change, methylation, acetylation, modification to amino acid side chains or the amino terminus, and changes in oxidation, disulfide-bond formation, etc.

“Sample” as used herein generally refers to a type of source or a state of a source, for example, a given cell type or tissue. The state of a source may be modified by certain treatments, such as by contacting the source with a chemical compound, before the source is used in the methods of the invention. It should be noted that protein interaction network data based on “a sample” does not necessarily comprise results obtained from a single experiment. Rather, to completely determine a protein interaction network, multiple experiments are often needed, and the combined results of which are used to construct the protein interaction network data for that particular sample.

By “semi-purified”, with respect to protein preparations, it is meant that the proteins have been previously separated from other cellular or viral proteins. For instance, in contrast to whole cell lysates, the proteins of reconstituted conjugation system, together with the substrate protein, can be present in the mixture to at least 50% purity relative to all other proteins in the mixture, more preferably are present at least 75% purity, and even more preferably are present at 90-95% purity.

The term “semi-purified cell extract” or, alternatively, “fractionated lysate”, as used herein, refers to a cell lysate which has been treated so as to substantially remove at least one component of the whole cell lysate, or to substantially enrich at least one component of the whole cell lysate. “Substantially remove”, as used herein, means to remove at least 10%, more preferably at least 50%, and still more preferably at least 80%, of the component of the whole cell lysate. “Substantially enrich”, as used herein, means to enrich by at least 10%, more preferably by at least 30%, and still more preferably at least about 50%, at least one component of the whole cell lysate compared to another component of the whole cell lysate. The term “semipurified cell extract” is also intended to include the lysate from a cell, when the cell has been treated so as to have substantially more, or substantially less, of a given component than a control cell. For example, a cell which has been modified (by, e.g., recombinant DNA techniques) to produce none (or very little) of a component of a signaling pathway, will, upon cell lysis, yield a semi-purified cell extract.

The terms “signal transduction,” “signaling,” “signal transduction pathway,” “signaling pathway,” etc. are used herein interchangeably and refer to the processing of physical or chemical signals from the cellular environment through the cell membrane, and may occur through one or more of several mechanisms, such as activation/inactivation of enzymes (such as proteases, or other enzymes which may alter phosphorylation patterns or other post-translational modifications), activation of ion channels or intracellular ion stores, effector enzyme activation via guanine nucleotide binding protein intermediates, formation of inositol phosphate, activation or inactivation of adenylyl cyclase, direct activation (or inhibition) of a transcriptional factor and/or activation, etc.

“Small molecule” as used herein, is meant to refer to a composition, which has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures comprising arrays of small molecules, often fungal, bacterial, or algal extracts, which can be screened with any of the assays of the invention.

“Solid support” or “carrier,” used interchangeably, refers to a material which is an insoluble matrix, and may (optionally) have a rigid or semi-rigid surface. Such materials may take the form of small beads, pellets, disks, chips, dishes, multi-well plates, wafers or the like, although other forms may be used. In some embodiments, at least one surface of the substrate will be substantially flat.

As applied to polypeptides, “substantial sequence identity” means that two mammalian peptide sequences, when optimally aligned, such as by the programs GAP or BESTFIT using default gap which share at least 90 percent sequence identity, preferably at least 95 percent sequence identity, more preferably at least 99 percent sequence identity or more. Preferably, residue positions which are not identical differ by conservative amino acid substitutions. For example, the substitution of amino acids having similar chemical properties such as charge or polarity are not likely to effect the properties of a protein. Examples include glutamine for asparagine or glutamic acid for aspartic acid.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

Overview

The current progression from genomics to proteomics is fueled by the realization that many properties of proteins (e.g., interactions, post-translational modifications) cannot be predicted from DNA sequence. The present invention provides a method useful to identify modified amino acid sites within peptide analytes. These modified amino acids are amino acids that incorporate conjugating groups including but not limited to those conjugating groups are that incorporated naturally by the cell, typically as post-translational modifications. Such conjugating groups include saccharide moieties, such as monosaccharides, disaccharides and polysaccharides. Such conjugating groups further include lipids and glycosaminoglycans. Other modified amino acids containing various types of conjugating groups can also be detected by the present method, including amino acids modified by iodination, bromination, nitration and sulfation, and particularly amino acids modified by phosphorylation. In certain preferred embodiments, the subject method is used to identify phosphate modified serine, threonine, tyrosine, histidine, arginine, lysine, cysteine, glutamic acid and aspartic acid residues, more preferably to identify phosphoserine, phosphothreonine and phosphotyrosine containing peptides.

The subject invention provides apparatus and methods for automating the use of mass spectroscopy for identifying post-translationally modified polypeptides. In particular, the subject method provides for automation of a process including affinity chromatography capture of post-translationally modified proteins, and processing the modified proteins for analysis by mass spectroscopy. Unlike the prior art methods which require conversion of the modified amino acid residue to another chemical entity which can be used to purify a particular peptide, the subject method is based on affinity capture by way of the originally modified amino acid residue after treatment of the peptide with agents that modify other residues in the peptide which might otherwise interfere with the affinity capture of the peptide.

The salient advantage of the subject method is that it can be incorporated in an automated system that reduces the amount of tedious manual labor associated with the traditional method of phosphopeptide analysis. Using methods taught in the prior art, the complete process generally takes at least 2 hours to carry out and requires significant vigilance on the part of the experimentalist. An experienced researcher can generally do no more than 3-4 runs in a day. An automated system (or a series of such systems) can dramatically increase the amount of samples processed per day since most human resource limits are eliminated. Other advantages include:

Efficiency and reproducibility are also increased as the automated components deliver consistent performance not possible with manual methods.

The automated system also allows for multiple column switching abilities. This multiplexing ability can dramatically increase the number of samples analyzed per day.

The incorporation of automated HPLC pumps in the automation process allows the use of gradient elution of the IMAC column, a process not possible by manual methods.

The amount of sample handling is reduced.

The subject method can be illustrated by the example of its use in identifying phosphorylated polypeptides. Phosphopeptides bind Fe(III) with high selectivity, so are amenable to affinity purification using Fe(III) immobilized metal-ion affinity chromatography (IMAC) techniques. However, the presence of hydroxyl and carboxyl groups in the sample peptides, e.g., due to a free carboxyl terminus and the presence of side chains such glutamic acid and aspartic acid, can reduce the efficiency of purification by contributing to non-specific binding to the metal column. Conversion of these side chains to neutral derivatives, such as by alkyl-esterification (which converts Glu and Asp to their neutral, alkyl ester derivatives, and also converts the C-terminal carboxyl group to an alkyl ester) can be used to reduce non-specific binding. The phosphate groups, if any, are not neutralized under the reaction conditions, and are accordingly still available for coordinating a metal ion. Thus, the resulting peptide mixture is contacted with a metal affinity column or resin which retains only peptides which bear the phosphate groups. The other peptides “flow through” the column. The phosphopeptides can then be eluted in a second step and analyzed by mass spectrometry, such as LC/MS/MS. Sequencing of the peptides can reveal both their identity and the site of phosphorylation.

To further illustrate, alkyl esters of free carboxyl groups in a peptide can be formed by reaction with alkyl halides and salts of the carboxylic acids, in an amide-type solvent, particularly dimethylformamide, in the presence of an iodine compound. In other embodiments, the reaction can be carried out with equimolecular amounts of an alkyl halide and a tertiary aliphatic amine.

In yet another embodiment, the method of the present invention can include esterification of the free carboxylic groups by reacting a salt of the carboxylic acid with a halogenated derivative of an aliphatic hydrocarbon, a cycloaliphatic hydrocarbon or an aliphatic hydrocarbon bearing a cyclic substituent in an aqueous medium, and in the presence of a phase transfer catalyst. By the expression “phase transfer catalyst” is intended a catalyst which transfers the carboxylate anion from the aqueous phase into the organic phase. The preferred catalysts for the process of the invention are the onium salts and more particularly quaternary ammonium and/or phosphonium salts.

The alkyl ester of the dipeptide is most preferably a methyl ester and may also be an ethyl ester or alkyl of up to about four carbon atoms such as propyl, isopropyl, butyl or isobutyl.

In still other embodiments, the carboxyl groups can be modified using reagents which are traditionally employed as carboxyl protecting groups or cross-coupling agents, such as 1,3-dicyclohexylcarbodiimide (DCC), 1,1′carbonyldiimidazole (CDI), 1-ethyl-3-(3-dimethylaminopropyl) carbodiimide hydrochloride (EDC), benzotriazol-1-yl-oxytris(dimethylamino) phosphonium hexafluorophosphate (BOP), and 1,3-Diisopropylcarbodiimide (DICD).

It will be appreciated by those skilled in the art that the subject method can be extended to other types of protein modifications, particularly those which result in modification(s) which change the protein's susceptibility to metal ion affinity purification in a manner dependent on the presence of the modified residues and which difference is enhanced by further chemical modification of other amino acid side chains and/or terminal groups of the protein. Exemplary post-translation modifications for which the subject method can used include glycosylation, acylation, methylation, phosphorylation, sulfation, prenylation, hydroxylation and carboxylation. For example, the automated analysis of glycopeptides could be accomplished by substituting a boronate-type column into the system. Alternatively, a thiol-containing column could be used to purify cysteine-containing peptides. As in the case of phosphorylation, the method can include steps for treating protein samples with agents that selectively react with certain groups that are typically found in peptides (e.g., sulfhydryl, amino, carboxy, hydroxyl groups and the like).

In certain embodiments, the proteins or protein mixtures are processed, e.g., cleaved either chemically or enzymatically, to reduce to the proteins to smaller peptides fragments. In certain preferred embodiments, the amide backbone of the proteins are cleaved through enzymatic digestion, preferably treatment of the proteins with an enzyme which produces a carboxy terminal lysine and/or arginine residue, such as selected from the group of trypsin, Arg-C and Lys-C, or a combination thereof. This digestion step may not be necessary, if the proteins are relatively small.

In certain embodiments, the reactants and reaction conditions can be selected such that differential isotopic labeling can be carried out across multiple different samples to generate substantially chemically identical, but isotopically distinguishable peptides. In this way, the source of particular samples can be encoded in the label. This technique can be used to quantitate differences in phosphorylation patterns and/or levels of phosphorylation between two or more samples. Merely to illustrate, the esterification reaction can be performed on one sample in the matter described above. In another sample, esterification is performed by deuterated or tritiated alkyl alcohols, e.g., D₃COD (D₄ methyl-alcohol), leading to the incorporation of three deuterium atoms instead of hydrogen atoms for each site of esterification. Likewise, ¹⁸O can be incorporated into peptides. The peptide mixtures from the two samples are then mixed and analyzed together, for example by LC/MS/MS. The phosphopeptides will be detected as light and heavy forms, and the relative ratio of peak intensities can be used to calculate the relative ratio of the phosphorylation in the two cases.

It can also be advantageous to perform one methyl-esterification reaction on the whole protein with methyl-alcohol for both samples. Subsequent to enzymatic digestion, one of the samples is then further esterified with D4 Methyl-alcohol. This leads to the incorporation of three deuterium atoms in each peptide rather than a variable number depending on the number of acidic residues in the peptide.

To complete the analysis, the sample may be further separated by reverse phase chromatography and on-line mass spectrometry analysis using both MS and MS/MS. To illustrate, the sequence of isolated peptides can be determined using tandem MS (MSn) techniques, and by application of sequence database searching techniques, the protein from which the sequenced peptide originated can be identified. In general, at least one peptide sequence derived from a protein will be characteristic of that protein and be indicative of its presence in the mixture. Thus, the sequences of the peptides typically provide sufficient information to identify one or more proteins present in a mixture.

Quantitative relative amounts of proteins in one or more different samples containing protein mixtures (e.g., biological fluids, cell or tissue lysates, etc.) can be determined using isotopic labeling as described above. In this method, each sample to be compared is treated with a different isotopically labeled reagent. The treated samples are then combined, preferably in equal amounts, and the proteins in the combined sample are enzymatically digested, if necessary, to generate peptides. As described above, peptides are isolated by affinity purification based on the post-translation modification of interest and analyzed by MS. The relative amounts of a given protein in each sample is determined by comparing relative abundance of the ions generated from any differentially labeled peptides originating from that protein. More specifically, the method can be applied to screen for and identify proteins which exhibit differential levels of modification in cells, tissue or biological fluids.

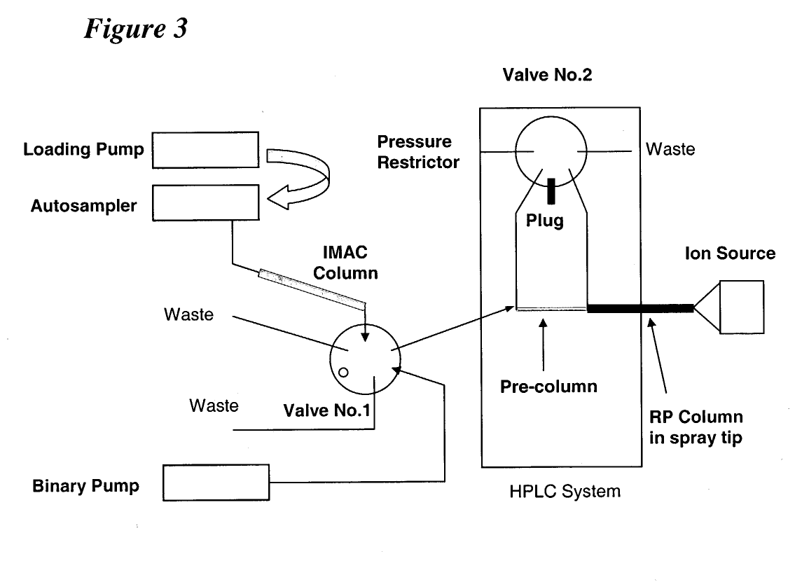

A schematic configuration of equipment which can be used to automate the subject method is shown in FIG. 3. Basic components include an autosampler, a loading pump, two 6-port valves, a binary pump, a pre-column, an IMAC column, and an ion source capable of interfacing with any commercially available mass spectrometer. The autosampler preferably has pretreatment capability and the ability to hold at least 6 reagent bottles for liquid handling capability. In the illustrate embodiment, the user is only required to prepare the samples and place them in the autosampler.

In operation, at the beginning of the process Valve No. 1 is at such position that solvent stream through the IMAC column goes directly to waste, while at the same time, solvent from the binary pump goes to the nano HPLC assembly. Valve No. 2 is at such position that flow from the binary pump is allowed to vent at the pressure restrictor, which generates back pressure for nanoliter per minute flow at the column tip.

When analysis starts, the autosampler injects condition buffers according to the sequence previously described. The sample solution is then injected. A wash solution is injected to remove peptides that non-specifically bind to the IMAC column. The last step of the process is elution when elution buffer is injected by the autosampler, Valve No. 1 switches to the position that allows eluting solvent containing phosphorylated peptides to be connected directly to the precolumn. At the same time, Valve No. 2 switches to a position such that solvent flow going through the precolumn is directed to waste.

After all phosphorylated peptides are loaded onto the precolumn, Valves No. 1 and No. 2 switch back to their original positions. A solvent composition gradient is started on the binary pump to complete the analysis.

The method of the present invention is useful for a variety of applications. For example, it permits the identification of enzyme substrates which are modified in response to different environmental cues provided to a cell. identification of those substrates, in turn, can be used to understand what intracellular signaling pathways are involved in any particular cellular response, as well as to identify the enzyme responsible for catalyzing the modification. To further illustrate, changes in phosphorylation states of substrate proteins can be used to identify kinases and/or phosphatases which are activated or inactivated in a manner dependent on particular cellular cues. In turn, those enzymes can be used as drug screening targets to find agents capable of altering their activity and, therefore, altering the response of the cell to particular environmental cues. So, for example, kinases and/or phosphatases which are activated in transformed (tumor) cells can be identified through their substrates, according to the subject method, and then used to develop anti-proliferative agents which are cytostatic or cytotoxic to the tumor cell.

In other embodiments, the present method can be used to identify a treatment that can modulate a modification of amino acid in a target protein without any knowledge of the upstream enzymes which produce the modified target protein. By comparing the level of a modification before and after certain treatments, one can identify the specific treatment that leads to a desired change in level of modification to one or more target proteins. To illustrate, one can screen a library of compounds, for example, small chemical compounds from a library, for their ability to induce or inhibit phosphorylation of a target polypeptide. While in other instances, it may be desirable to screen compounds for their ability to induce or inhibit the dephosphorylation of a target polypeptide (i.e., by a phosphatase).

Similar treatments are not limited to small chemical compounds. For example, a large number of known growth factors, cytokines, hormones and any other known agents known to be able to modulate post-translational modifications are also within the scope of the invention.

In addition, treatments are not limited to chemicals. Many other environmental stimuli are also known to be able to cause post-translational modifications. For example, osmotic shock may activate the p38 subfamily of MAPK and induce the phosphorylation of a number of downstream targets. Stress, such as heat shock or cold shock, many activate the JNK/SAPK subfamily of MAPK and induce the phosphorylation of a number of downstream targets. Other treatments such as pH change may also stimulate signaling pathways characterized by post-translational modification of key signaling components.

In another respect, the instant invention also provides a means to characterize the effect of certain treatments, i.e., identifying the specific post-translational modification on specific polypeptides as a result of the treatment.

To illustrate, one may wish to identify the effect of treating cells with a growth factor. More specifically, one may desire to identify the specific signal transduction pathways involved downstream of a growth factor. By comparing post-translational modification levels of certain candidate polypeptides before and after the growth factor treatment, one can use the method of the instant invention to determine precisely what downstream signaling pathways of interest are activated or down regulated. This in turn also leads to the identification of potential drug screen targets if such signaling pathways are to be modulated.

In connection with those methods, the instant invention also provides a method for conducting a drug discovery business, comprising: i) by suitable methods mentioned above, determining the identity of a compound that modulates a modification of amino acid in a target polypeptide; ii) conducting therapeutic profiling of the compound identified in step i), or further analogs thereof, for efficacy and toxicity in animals; and, iii) formulating a pharmaceutical preparation including one or more compounds identified in step ii) as having an acceptable therapeutic profile. Such business method can be further extended by including an additional step of establishing a distribution system for distributing the pharmaceutical preparation for sale, and may optionally include establishing a sales group for marketing the pharmaceutical preparation.

The instant invention also provides a business method comprising: i) by suitable methods mentioned above, determining the identity of a compound that modulates a modification of amino acid in a target polypeptide; ii) licensing, to a third party, the rights for further drug development of compounds that alter the level of modification of the target polypeptide.

The instant invention also provides a business method comprising: i) by suitable methods mentioned above, determining the identity of the polypeptide and the nature of the modification induced by the treatment; ii) licensing, to a third party, the rights for further drug development of compounds that alter the level of modification of the polypeptide.

The following sections describe in detail about certain aspects of the invention.

Affinity Capture of Polypeptide Samples

Polypeptide separation and isolation schemes can achieved based on differences in the molecular properties such as size, charge and solubility. Protocols based on these parameters include SDS-PAGE (SDS-PolyAcrylamide Gel Electrophoresis), size exclusion chromatography, ion exchange chromatography, differential precipitation and the like. SDS-PAGE is well-known in the art of biology, and will not be described here in detail. See Molecular Cloning A Laboratory Manual, 2nd Ed., ed. by Sambrook, Fritsch and Maniatis (Cold Spring Harbor Laboratory Press: 1989).

Size exclusion chromatography, otherwise known as gel filtration or gel permeation chromatography, relies on the penetration of macromolecules in a mobile phase into the pores of stationary phase particles. Differential penetration is a function of the hydrodynamic volume of the particles. Accordingly, under ideal conditions the larger molecules are excluded from the interior of the particles while the smaller molecules are accessible to this volume and the order of elution can be predicted by the size of the polypeptide because a linear relationship exists between elution volume and the log of the molecular weight. Size exclusion chromatographic supports based on cross-linked dextrans e.g. SEPHADEX.RTM., spherical agarose beads e.g. SEPHAROSE.RTM. (both commercially available from Pharmacia AB. Uppsala, Sweden), based on cross-linked polyacrylamides e.g. BIO-GEL.RTM. (commercially available from BioRad Laboratories, Richmond, Calif.) or based on ethylene glycol-methacrylate copolymer e.g. TOYOPEARL HW65S (commercially available from ToyoSoda Co., Tokyo, Japan) are useful in the practice of this invention.

Precipitation methods are predicated on the fact that in crude mixtures of polypeptides the solubilities of individual polypeptides are likely to vary widely. Although the solubility of a polypeptide in an aqueous medium depends on a variety of factors, for purposes of this discussion it can be said generally that a polypeptide will be soluble if its interaction with the solvent is stronger than its interaction with polypeptide molecules of the same or similar kind. Without wishing to be bound by any particular mechanistic theory describing precipitation phenomena, it is nonetheless believed that the interaction between a polypeptide and water molecules occur by hydrogen bonding with several types of charged groups, and electrostatically as dipoles with uncharged groups, and that precipitants such as salts of monovalent cations (e.g., ammonium sulfate) compete with polypeptides for water molecules, thus at high salt concentrations, the polypeptides become “dehydrated” reducing their interaction with the aqueous environment and increasing the aggregation with like or similar polypeptides resulting in precipitation from the medium.

One form of affinity capture reagent can be used in ion exchange chromatography, which involves the interaction of charged functional groups in the sample with ionic functional groups of opposite charge on an adsorbent surface. Two general types of interaction are known. Anionic exchange chromatography mediated by negatively charged amino acid side chains (e.g. aspartic acid and glutamic acid) interacting with positively charged surfaces and cationic exchange chromatography mediated by positively charged amino acid residues (e.g. lysine and arginine) interacting with negatively charged surfaces.

More recently affinity chromatography and hydrophobic interaction chromatography techniques have been developed to supplement the more traditional size exclusion and ion exchange chromatographic protocols. Affinity chromatography relies on the interaction of the polypeptide with an immobilized ligand. The ligand can be specific for the particular polypeptide of interest in which case the ligand is a substrate, substrate analog, inhibitor or antibody. Alternatively, the ligand may be able to react with a number of polypeptides. Such general ligands as adenosine monophosphate, adenosine diphosphate, nicotine adenine dinucleotide or certain dyes may be employed to recover a particular class of polypeptides. One of the least biospecific of the affinity chromatographic approaches is immobilized metal affinity chromatography (IMAC), also referred to as metal chelate chromatography. IMAC introduced by Porath et al.(Nature 258:598-99(1975) involves chelating a metal to a solid support and then forming a complex with electron donor amino acid residues on the surface of a polypeptide to be separated.

Hydrophobic interaction chromatography was first developed following the observation that polypeptides could be retained on affinity gels which comprised hydrocarbon spacer arms but lacked the affinity ligand. Although in this field the term hydrophobic chromatography is sometimes used, the term hydrophobic interaction cluomatography (HIC) is preferred because it is the interaction between the solute and the gel that is hydrophobic not the chromatographic procedure. Hydrophobic interactions are strongest at high ionic strength, therefore, this form of separation is conveniently performed following salt precipitations or ion exchange procedures. Elution from HIC supports can be effected by alterations in solvent, pH, ionic strength, or by the addition of chaotropic agents or organic modifiers, such as ethylene glycol. A description of the general principles of hydrophobic interaction chromatography can be found in U.S. Pat. No. 3,917,527 and in U.S. Pat. No. 4,000,098. The application of HIC to the purification of specific polypeptides is exemplified by reference to the following disclosures: human growth hormone (U.S. Pat. No. 4,332,717), toxin conjugates (U.S. Pat. No. 4,771,128), antihemolytic factor (U.S. Pat. No. 4,743,680), tumor necrosis factor (U.S. Pat. No. 4,894,439), interleukin-2 (U.S. Pat. No. 4,908,434), human lymphotoxin (U.S. Pat. No. 4,920,196) and lysozyme species (Fausnaugh, J. L. and F. E. Regnier, J. Chromatog. 359:131-146 (1986)).

The principles of IMAC are generally appreciated. It is believed that adsorption is predicated on the formation of a metal coordination complex between a metal ion, immobilized by chelation on the adsorbent matrix, and accessible electron donor amino acids on the surface of the polypeptide to be bound. The metal-ion microenvironment including, but not limited to, the matrix, the spacer arm, if any, the chelating ligand, the metal ion, the properties of the surrounding liquid medium and the dissolved solute species can be manipulated by the skilled artisan to affect the desired fractionation.

Not wishing to be bound by any particular theory as to mechanism, it is further believed that the more important amino acid residues in terms of binding are histidine, tryptophan and probably cysteine. Since one or more of these residues are generally found in polypeptides, one might expect all polypeptides to bind to IMAC columns. However, the residues not only need to be present but also accessible (e.g., oriented on the surface of the polypeptide) for effective binding to occur. Other residues, for example poly-histidine tails added to the amino terminus or carboxyl terminus of polypeptides, can be engineered into the recombinant expression systems by following the protocols described in U.S. Pat. No. 4,569,794.

The nature of the metal and the way it is coordinated on the column can also influence the strength and selectivity of the binding reaction. Matrices of silica gel, agarose and synthetic organic molecules such as polyvinyl-methacrylate co-polymers can be employed. The matrices preferably contain substituents to promote chelation. Substituents such as iminodiacetic acid (IDA) or its tris (carboxymethyl) ethylene diamine (TED) can be used. IDA is preferred. A particularly useful IMAC material is a polyvinyl methacrylate co-polymer substituted with IDA available commercially, e.g., as TOYOPEARL AF-CHELATE 650M (ToyoSoda Co.; Tokyo. The metals are preferably divalent members of the first transition series through to zinc, although Co⁺⁺, Ni⁺⁺, Cd⁺⁺ and Fe⁺⁺⁺ can be used. An important selection parameter is, of course, the affinity of the polypeptide to be purified for the metal. Of the four coordination positions around these metal ions, at least one is occupied by a water molecule which is readily replaced by a stronger electron donor such as a histidine residue at slightly alkaline pH.

In practice the IMAC column is “charged” with metal by pulsing with a concentrated metal salt solution followed by water or buffer. The column often acquires the color of the metal ion (except for zinc). Often the amount of metal is chosen so that approximately half of the column is charged. This allows for slow leakage of the metal ion into the non-charged area without appearing in the eluate. A pre-wash with intended elution buffers is usually carried out. Sample buffers may contain salt up to 1M or greater to minimize nonspecific ion-exchange effects. Adsorption of polypeptides is maximal at higher pHs. Elution is normally either by lowering of pH to protonate the donor groups on the adsorbed polypeptide, or by the use of stronger complexing agent such as imidazole, or glycine buffers at pH 9. In these latter cases the metal may also be displaced from the column. Linear gradient elution procedures can also be beneficially employed.

As mentioned above, IMAC is particularly useful when used in combination with other polypeptide fractionation techniques. That is to say it is preferred to apply IMAC to material that has been partially fractionated by other protein fractionation procedures. A particularly useful combination chromatographic protocol is disclosed in U.S. Pat. No. 5,252,216 granted Oct. 12, 1993, the contents of which are incorporated herein by reference. It has been found to be useful, for example, to subject a sample of conditioned cell culture medium to partial purification prior to the application of IMAC. By the term “conditioned cell culture medium” is meant a cell culture medium which has supported cell growth and/or cell maintenance and contains secreted product. A concentrated sample of such medium is subjected to one or more polypeptide purification steps prior to the application of a IMAC step. The sample may be subjected to ion exchange chromatography as a first step. As mentioned above various anionic or cationic substituents may be attached to matrices in order to form anionic or cationic supports for chromatography. Anionic exchange substituents include diethylaminoethyl (DEAE), quaternary aminoethyl (QAE) and quaternary amine (Q) groups. Cationic exchange substituents include carboxymethyl (CM), sulfoethyl (SE), sulfopropyl (SP), phosphate (P) and sulfonate (S). Cellulosic ion exchange resins such as DE23, DE32, DE52, CM-23, CM-32 and CM-52 are available from Whatman Ltd. Maidstone, Kent, U.K. SEPHADEX.RTM.-based and cross-linked ion exchangers are also known. For example, DEAE-, QAE-, CM-, and SP-dextran supports under the tradename SEPHADEX.RTM. and DEAE-, Q-, CM-and S-agarose supports under the tradename SEPHAROSE.RTM. are all available from Pharmacia AB. Further both DEAE and CM derivatized ethylene glycol-methacrylate copolymer such as TOYOPEARL DEAE-650S and TOYOPEARL CM-650S are available from Toso Haas Co., Philadelphia, Pa. Because elution from ionic supports sometimes involves addition of salt and IMAC may be enhanced under increased salt concentrations. The introduction of a IMAC step following an ionic exchange chromatographic step or other salt mediated purification step may be employed. Additional purification protocols may be added including but not necessarily limited to HIC, further ionic exchange chromatography, size exclusion chromatography, viral inactivation, concentration and freeze drying.

Hydrophobic molecules in an aqueous solvent will self-associate. This association is due to hydrophobic interactions. It is now appreciated that macromolecules such as polypeptides have on their surface extensive hydrophobic patches in addition to the expected hydrophilic groups. HIC is predicated, in part, on the interaction of these patches with hydrophobic ligands attached to chromatographic supports. A hydrophobic ligand coupled to a matrix is variously referred to herein as an HIC support, HIC gel or HIC column. It is further appreciated that the strength of the interaction between the polypeptide and the HIC support is not only a function of the proportion of non-polar to polar surfaces on the polypeptide but by the distribution of the non-polar surfaces as well.

A number of matrices may be employed in the preparation of HIC columns, the most extensively used is agarose. Silica and organic polymer resins may be used. Useful hydrophobic ligands include but are not limited to alkyl groups having from about 2 to about 10 carbon atoms, such as a butyl, propyl, or octyl; or aryl groups such as phenyl. Conventional HIC products for gels and columns may be obtained commercially from suppliers such as Pharmacia LKB AB, Uppsala, Sweden under the product names butyl-SEPHAROSE.RTM., phenyl-SEPHAROSE.RTM. CL-4B, octyl-SEPHAROSE.RTM. FF and phenyl-SEPHAROSE.RTM. FF; Tosoh Corporation, Tokyo, Japan under the product names TOYOPEARL Butyl 650, Ether-650, or Phenyl-650 (FRACTOGEL TSK Butyl-650) or TSK-GEL phenyl-5PW; Miles-Yeda, Rehovot, Israel under the product name ALKYL-AGAROSE, wherein the alkyl group contains from 2-10 carbon atoms, and J. T. Baker, Phillipsburg, N.J. under the product name BAKERBOND WP-HI-propyl.

Ligand density is an important parameter in that it influences not only the strength of the interaction but the capacity of the column as well. The ligand density of the commercially available phenyl or octyl phenyl gels is on the order of 40 μM/ml gel bed. Gel capacity is a function of the particular polypeptide in question as well pH, temperature and salt concentration but generally can be expected to fall in the range of 3-20 mg/ml of gel.

The choice of a particular gel can be determined by the skilled artisan. In general the strength of the interaction of the polypeptide and the HIC ligand increases with the chain length of the of the alkyl ligands but ligands having from about 4 to about 8 carbon atoms are suitable for most separations. A phenyl group has about the same hydrophobicity as a pentyl group, although the selectivity can be quite different owing to the possibility of pi-pi interaction with aromatic groups on the polypeptide.

Adsorption of the polypeptides to a HIC column is favored by high salt concentrations, but the actual concentrations can vary over a wide range depending on the nature of the polypeptide and the particular HIC ligand chosen. Various ions can be arranged in a so-called soluphobic series depending on whether they promote hydrophobic interactions (salting-out effects) or disrupt the structure of water (chaotropic effect) and lead to the weakening of the hydrophobic interaction. Cations are ranked in terms of increasing salting out effect as Ba⁺⁺<Ca⁺⁺<Mg⁺⁺<Li⁺<Cs⁺<Na⁺<K⁺<Rb⁺<NH₄⁺. While anions may be ranked in terms of increasing chaotropic effect as PO₄⁻⁻⁻<SO₄⁻⁻<CH₃COO⁻<Cl⁻<Br⁻<NO₃⁻<ClO₄⁻<I⁻<SCN⁻.

Accordingly, salts may be formulated that influence the strength of the interaction as given by the following relationship:

Na₂SO₄>NaCl>(NH₄)₂SO₄>NH₄Cl>NaBr>NaSCN

In general, salt concentrations of between about 0.75 and about 2M ammonium sulfate or between about 1 and 4M NaCl are useful.

The influence of temperature on IAIC separations is not simple, although generally a decrease in temperature decreases the interaction. However, any benefit that would accrue by increasing the temperature must also be weighed against adverse effects such an increase may have on the activity of the polypeptide.

Elution, whether stepwise or in the form of a gradient, can be accomplished in a variety of ways: (a) by changing the salt concentration, (b) by changing the polarity of the solvent or (c) by adding detergents. By decreasing salt concentration adsorbed polypeptides are eluted in order of increasing hydrophobicity. Changes in polarity may be affected by additions of solvents such as ethylene glycol or (iso)propanol thereby decreasing the strength of the hydrophobic interactions. Detergents function as displacers of polypeptides and have been used primarily in connection with the purification of membrane polypeptides.

When the eluate resulting from HIC is subjected to further ion exchange chromatography, both anionic and cationic procedures may be employed.

As mentioned above, gel filtration chromatography affects separation based on the size of molecules. It is in effect a form of molecular sieving. It is desirable that no interaction between the matrix and solute occur, therefore, totally inert matrix materials are preferred. It is also desirable that the matrix be rigid and highly porous. For large scale processes rigidity is most important as that parameter establishes the overall flow rate. Traditional materials such as crosslinked dextran or polyacrylamide matrices, commercially available as, e.g., SEPHADEX.RTM. and BIOGEL.RTM., respectively, were sufficiently inert and available in a range of pore sizes, however these gels were relatively soft and not particularly well suited for large scale purification. More recently, gels of increased rigidity have been developed (e.g. SEPHACRYL.RTM., ULTROGEL.RTM., FRACTOGEL.RTM. and SUPEROSE.RTM.). All of these materials are available in particle sizes which are smaller than those available in traditional supports so that resolution is retained even at higher flow rates. Ethylene glycol-methacrylate copolymer matrices, e.g., such as the TOYOPEARL HW series matrices (Toso Haas) are preferred.

Phosphoproteins can be isolated using IMAC as described above. However, they can also be isolated by other means. Specifically, phosphoproteins with phosphorylated tyrosine residues can be isolated with phospho-tyrosine specific antibodies, which may be affixed to affinity columns using well-known routine procedure. Likewise, phospho-serine/threonine specific antibodies can be used to isolate phosphoproteins with phosphorylated serine/threonine residues. Many of these antibodies are available as affinity purified forms, either as monoclonal antibodies or antisera or mouse ascites fluid. For example, phospho-Tyrosine monoclonal antibody (P-Tyr102) is a high-affinity IgG1 phospho-tyrosine antibody clone that is produced and characterized by Cell Signaling Technology (Beverly, Mass.). As determined by ELISA, P-Tyr-102 (Cat. No. 9416) binds to a larger number of phospho-tyrosine containing peptides in a manner largely independent of the surrounding amino acid sequences, and also interacts with a broader range of phospho-tyrosine containing polypeptides as indicated by 2D-gel Western analysis. P-Tyr-102 is highly specific for phospho-Tyr in peptides/proteins, shows no cross-reactivity with the corresponding nonphosphorylated peptides and does not react with peptides containing phospho-Ser or phospho-Thr instead of phospho-Tyr. It is expected that P-Tyr-102 will react with peptides/proteins containing phospho-Tyr from all species.

Phospho-threonine antibodies are also available. For example, Cell Signaling Technology also offer an affinity-purified rabbit polyclonal phospho-threonine antibody (P-Thr-Polyclonal, Cat. No. 9381) which binds threonine-phosphorylated sites in a manner largely independent of the surrounding amino acid sequence. It recognizes a wide range of threonine-phosphorylated peptides in ELISA and a large number of threonine-phosphorylated polypeptides in 2D analysis. It is specific for peptides/proteins containing phospho-Thr and shows no cross-reactivity with corresponding nonphosphorylated sequences. Phospho-Threonine Antibody (P-Thr-Polyclonal) does not cross-react with sequences containing either phospho-Tyrosine or phospho-Serine. It is expected that this antibody will react with threonine-phosphorylated peptides/proteins regardless of species of origin. Upstate Biotechnology (Lake Placid, N.Y.) also provides an anti-phosphoserine/threonine antibody with broad immunoreactivity for polypeptides containing phosphorylated serine and phosphorylated threonine residues.

Many other similar products are also available on the market. These antibodies can be readily coupled to supporting matrix materials to generate affinity columns according to standard molecular biology protocols (for details and general means of antibody production, see Using Antibodies: A Laboratory Manual: Portable Protocol NO. I, Harlow and Lane, Cold Spring Harbor Laboratory Press: 1998; also see Antibodies: A Laboratory Manual, edited by Harlow and Lane, Cold Spring Harbor Laboratory Press: 1988).

A similar approach can be applied towards the isolation of any specific polypeptide, against which specific antibodies are available.

Isolation of membrane-associated polypeptides can be carried out using appropriate methods as described above (for example, hydrophobic interaction chromatography). Alternatively, it can be performed with other standard molecular biology protocols. See, for example, Molecular Cloning A Laboratory Manual, 2nd Ed., ed. by Sambrook, Fritsch and Maniatis (Cold Spring Harbor Laboratory Press: 1989); B. Perbal, A Practical Guide To Molecular Cloning (1984); the treatise, Methods In Enzymology (Academic Press, Inc., N.Y.); Methods In Enzymology, Vols. 154 and 155 (Wu et al. eds.), Immunochemical Methods In Cell And Molecular Biology (Mayer and Walker, eds., Academic Press, London, 1987).

For example, cells can be lysed in appropriate buffers and the membrane portions can be isolated by centrifugation. Depending on particular cases, cells preferably can be lysed in hypotonic buffer by homogenization. Cell debris and nuclei can then be removed by low speed centrifugation, followed by high speed centrifugation (such as under centrifugation conditions of 100,000×g or more) to pellet membrane portions. Membrane polypeptides can then be extracted by organic solvents such as chloroform and methanol.

Alternatively, membrane polypeptides can be isolated by extraction of membrane portions with extraction buffer containing detergents. Depending on specific occasions, the detergent used can be SDS or other ionic or non-ionic detergents. Different choices of detergent or extraction buffer in general may facilitate global non-biased extraction of membrane polypeptides or isolation of specific membrane polypeptides of interest. The reduced complexity of polypeptide mixtures resulting from the use of specific extraction protocols may be beneficial for the following digestion, separation, and analysis procedures.

One method of isolating hydrophobic membrane proteins is strong cation exchange (SCX) chromatography. Strong cation exchange (SCX) chromatography is particularly suited for isolating/purifying hydrophobic proteins, such as membrane proteins. Many SCX chromatographic columns are commercially available. For illustration purpose only, details regarding one type of SCX column, the PolySulfoethyl Aspartamide Strong Cation Exchange Columns manufactured by The Nest Group, Inc. (45 Valley Road, Southborough, Mass.), are described below. It is to be understood that the recommendations below are by no means limiting in any respect. Many other commercial SCX columns are also available, and should be used according to the recommendation of respective manufacturers.

According to the manufacturer, aspartamide cation exchange chemistries are some of the best materials available for the HPLC separation of peptides. These are wide-pore (300 Å) silica packings with a bonded coating of hydrophilic, sulfoethyl anionic polymer. With the PolySULFOETHYL Aspartamide SCX column, mobile phase modifiers can be used to help improve peptide solubility or to immediate the interaction between peptide and stationary phase. By varying the pH, ionic strength or organic solvent concentration in the mobile phase, chromatographic selectivity can be significantly enhanced. For more strongly hydrophobic peptides, a non-ionic surfactant (at a concentration below its CMC) and/or acetonitrile or n-propanol as mobile phase modifiers, can substantially improve resolution and recovery over conventional reverse phase methods. Additional selectivity can be obtained by simply changing the slope of the KCl or (NH₄)₂SO₄ gradient.

Using this column at pH 3 is better for retention of neutral to slightly acidic peptides. Use of a higher pH may be considered for basic hydrophobic peptides. The addition of MeCN or propanol to the A&B solvents (see below) changes the mechanism of separation and results in a separation based not only on positive charge, but also on hydrophobicity.

These columns are quite useful for neuropeptides, growth factors, CNBr peptide fragments, and synthetic peptides as a complement to RPC (Reverse Phase Chromatography), or to remove organic reagents from peptide samples which would cause smearing on a RPC column.

The operating conditions for these applications for an analytical column are:

Buffer A: 5 mM K—PO₄+25% MeCN;

Buffer B: 5 mM K—PO₄+25% MeCN+300-500 mM KCl;

Linear gradient, 30 min at 1 ml/min.

The peptides are retained on the column by the positive charge of at least the terminus amino and elute by total charge, charge distribution and hydrophobicity. If the peptide does not stick to the column, prepare the peptide in a small amount of buffer, or decrease the concentration of organic in the A&B solvents to 5 or 10%. Organic solvent concentration is empirically determined and n-propanol can be substituted for MeCN for more hydrophobic species.

Since the total binding capacity of these columns is on the order of 100 mg/gm of packing (for nonresolved materials) there will be a considerable Donan effect present. It will be necessary to have the sample in 5-15 mM of salt or buffer to prevent exclusion from the column. Additionally, the gradient at the outlet of the column will be much more concave than that observed on the chart paper. It is recommended that an upper load limit of 1 milligram for an analytical column. For a guard column used as a methods development column, a load limit of one-tenth of a milligram is recommended.

Flow rates of 0.7 to 1.0 ml/min with a 30 minutes gradient should be used for the analytical column. If using the 4.6×20 mm guard column as a methods development column, gradient times should be shortened to 8-10 min at the same flow rate since the void volume is only 0.3 ml. The semiprep columns, 9.4 mm ID, require flow rates and equilibration volumes 4×that of the analytical columns.

Typically, for the first run, equilibrate the analytical column in the high salt (or final pH) solution (at least 25 ml, or for a guard column used as a methods development column use 8 ml, or on the semiprep column use 100 ml), and inject the sample under these isocratic conditions to observe the elution profile. The protein should elute at the void volume. Then equilibrate the column in low salt (or low pH if doing a pH gradient) conditions and run the gradient to the final conditions. Comparison of the chromatograms will assure that the proteins will elute in a predictable fashion. To decrease elution times increase the salt concentration (in a convex or step manner), increase the pH, or shorten the equilibration times between gradient runs. Exposure to a pH above 7 should be avoided since this will affect the silica support and will shorten column life, as will temperatures above 45° C. For buffer gradients, phosphate or bis-tris are good buffers to use since they allow monitoring in the low UV range. For salt gradients, acetate salts are frequently used. However, it may be necessary to use sulfate or chloride if the buffering capacity of acetate is undesirable or if the absorbance is to be monitored below 235 nm. When chloride has been used for salt gradient elution, flush the column with at least 30 ml of deionized water at the end of the day to prevent corrosion. If a denaturant such as 4M urea is used in the mobile phase to increase the accessibility of the ionizable groups, be sure to have a silica saturator column in line in front of the injector, to minimize attack of the silica on the ion exchange column.

New columns should be condition before use, preferably according to the following protocol. Specifically, columns are filled with methanol when shipped so the (analytical) column should be flushed with at least 40 ml water before elution with salt solution to prevent precipitation. The hydrophilic coating imbibes a layer of water. The resultant swelling of the coating leads to a slight and irreversible increase in the column back pressure. Some additional swelling occurs with extended use of the column. Since the swelling increases the surface area of the coating, the capacity of the column for proteins increases as well. Thus, retention times may increase by up to 10%. This process should be hastened by eluting the column with a strong buffer for at least one hour prior to its initial use. A convenient solution to use is 0.2 M monosodium phosphate +0.3 M sodium acetate.

The conditioning process is reversed by exposing the column to pure organic solvents. Accordingly, to minimize the time to start the column after a 1-2 day storage, the column should be flushed with at least 40 ml of deionized water (not methanol), and the ends should be plugged. For extended storage it is recommended that a 100% methanol storage be used to prevent bacterial growth and contamination. Exercise care when using organic solvents to prevent precipitation of salts.

It is recommended that a new column be conditioned with two injections of an inexpensive protein (e.g. BSA) before it is used to analyze very dilute or expensive samples since new HPLC columns sometimes absorb small quantities of proteins in a nonspecific manner. The sintered metal frits have been implicated in this process. Fortunately these sites are quickly saturated. Mobile phases should be filtered before use, as should samples. Failure to do so may cause the inlet frit to plug. A guard column, P410-2SEA, will prevent damage to the analytical or preparative columns. Use of 0.1% TFA or high concentrations of formic acid in the mobile phase is not recommended.

For use in normal phase and HILIC polarity, the following should be taken into consideration. By adding even more organic solvent to the mobile phase, these columns offer enough flexibility so that they may be used in a normal or Hydrophilic Interaction (HILIC) mode. Here, more polar peptides having little or no retention under conventional reverse-phase or even ion-exchange conditions are retained, and very hydrophobic peptides may have enhanced solubility and thus chromatograph better. There are two approaches to this mode: 1) using isocratic HILIC conditions or 2) using a sodium perchlorate gradient. The key to achieving HILIC conditions is to use greater than 70% organic solvent with the SCX column. Care should be taken to assure solubility of salts under these conditions.

Mass Spectrometers, Detection Methods and Sequence Analysis

In certain embodiments, the isolated proteins are subjected to protease digestion followed by mass spectrometry. During the past decade, new techniques in mass spectrometry have made it possible to accurately measure with high sensitivity the molecular weight of peptides and intact proteins. These techniques have made it much easier to obtain accurate peptide masses of a protein for use in databases searches. Mass spectrometry provides a method, of protein identification that is both very sensitive (10 fmol-1 pmol) and very rapid when used in conjunction with sequence databases. Advances in protein and DNA sequencing technology are resulting in an exponential increase in the number of protein sequences available in databases. As the size of DNA and protein sequence databases grows, protein identification by correlative peptide mass matching has become an increasingly powerful method to identify and characterize proteins.

Mass Spectrometry

Mass spectrometry, also called mass spectroscopy, is an instrumental approach that allows for the gas phase generation of ions as well as their separation and detection. The five basic parts of any mass spectrometer include: a vacuum system; a sample introduction device; an ionization source; a mass analyzer; and an ion detector. A mass spectrometer determines the molecular weight of chemical compounds by ionizing, separating, and measuring molecular ions according to their mass-to-charge ratio (m/z). The ions are generated in the ionization source by inducing either the loss or the gain of a charge (e.g. electron ejection, protonation, or deprotonation). Once the ions are formed in the gas phase they can be electrostatically directed into a mass analyzer, separated according to mass and finally detected. The result of ionization, ion separation, and detection is a mass spectrum that can provide molecular weight or even structural information.

A common requirement of all mass spectrometers is a vacuum. A vacuum is necessary to permit ions to reach the detector without colliding with other gaseous molecules. Such collisions would reduce the resolution and sensitivity of the instrument by increasing the kinetic energy distribution of the ion's inducing fragmentation, or preventing the ions from reaching the detector. In general, maintaining a high vacuum is crucial to obtaining high quality spectra.

The sample inlet is the interface between the sample and the mass spectrometer. One approach to introducing sample is by placing a sample on a probe which is then inserted, usually through a vacuum lock, into the ionization region of the mass spectrometer. The sample can then be heated to facilitate thermal desorption or undergo any number of high-energy desorption processes used to achieve vaporization and ionization.

Capillary infusion is often used in sample introduction because it can efficiently introduce small quantities of a sample into a mass spectrometer without destroying the vacuum. Capillary columns are routinely used to interface the ionization source of a mass spectrometer with other separation techniques including gas chromatography (GC) and liquid chromatography (LC). Gas chromatography and liquid chromatography can serve to separate a solution into its different components prior to mass analysis. Prior to the 1980's, interfacing liquid chromatography with the available ionization techniques was unsuitable because of the low sample concentrations and relatively high flow rates of liquid chromatography. However, new ionization techniques such as electrospray were developed that now allow LC/MS to be routinely performed. One variation of the technique is that high performance liquid chromatography (HPLC) can now be directly coupled to mass spectrometer for integrated sample separation/preparation and mass spectrometer analysis.

In terms of sample ionization, two of the most recent techniques developed in the mid 1980's have had a significant impact on the capabilities of Mass Spectrometry: Electrospray Ionization (ESI) and Matrix Assisted Laser Desorption/Ionization (MALDI). ESI is the production of highly charged droplets which are treated with dry gas or heat to facilitate evaporation leaving the ions in the gas phase. MALDI uses a laser to desorb sample molecules from a solid or liquid matrix containing a highly UV-absorbing substance.

The MALDI-MS technique is based on the discovery in the late 1980s that an analyte consisting of, for example, large nonvolatile molecules such as proteins, embedded in a solid or crystalline “matrix” of laser light-absorbing molecules can be desorbed by laser irradiation and ionized from the solid phase into the gaseous or vapor phase, and accelerated as intact molecular ions towards a detector of a mass spectrometer. The “matrix” is typically a small organic acid mixed in solution with the analyte in a 10,000:1 molar ratio of matrix/analyte. The matrix solution can be adjusted to neutral pH before mixing with the analyte.

The MALDI ionization surface may be composed of an inert material or else modified to actively capture an analyte. For example, an analyte binding partner may be bound to the surface to selectively absorb a target analyte or the surface may be coated with a thin nitrocellulose film for nonselective binding to the analyte. The surface may also be used as a reaction zone upon which the analyte is chemically modified, e.g., CNBr degradation of protein. See Bai et al, Anal. Chem. 67, 1705-1710 (1995).

Metals such as gold, copper and stainless steel are typically used to form MALDI ionization surfaces. However, other commercially-available inert materials (e.g., glass, silica, nylon and other synthetic polymers, agarose and other carbohydrate polymers, and plastics) can be used where it is desired to use the surface as a capture region or reaction zone. The use of Nation and nitrocellulose-coated MALDI probes for on-probe purification of PCR-amplified gene sequences is described by Liu et al., Rapid Commun. Mass Spec. 9:735-743 (1995). Tang et al. have reported the attachment of purified oligonucleotides to beads, the tethering of beads to a probe element, and the use of this technique to capture a complimentary DNA sequence for analysis by MALDI-TOF MS (reported by K. Tang et al., at the May 1995 TOF-MS workshop, R. J. Cotter (Chairperson); K. Tang et al., Nucleic Acids Res. 23, 3126-3131, 1995). Alternatively, the MALDI surface may be electrically- or magnetically activated to capture charged analytes and analytes anchored to magnetic beads respectively.

Aside from MALDI, Electrospray Ionization Mass Spectrometry (ESI/MS) has been recognized as a significant tool used in the study of proteins, protein complexes and biomolecules in general. ESI is a method of sample introduction for mass spectrometric analysis whereby ions are formed at atmospheric pressure and then introduced into a mass spectrometer using a special interface. Large organic molecules, of molecular weight over 10,000 Daltons, may be analyzed in a quadrupole mass spectrometer using ESI.

In ESI, a sample solution containing molecules of interest and a solvent is pumped into an electrospray chamber through a fine needle. An electrical potential of several kilovolts may be applied to the needle for generating a fine spray of charged droplets. The droplets may be sprayed at atmospheric pressure into a chamber containing a heated gas to vaporize the solvent. Alternatively, the needle may extend into an evacuated chamber, and the sprayed droplets are then heated in the evacuated chamber. The fine spray of highly charged droplets releases molecular ions as the droplets vaporize at atmospheric pressure. In either case, ions are focused into a beam, which is accelerated by an electric field, and then analyzed in a mass spectrometer.

Because electrospray ionization occurs directly from solution at atmospheric pressure, the ions formed in this process tend to be strongly solvated. To carry out meaningful mass measurements, solvent molecules attached to the ions should be efficiently removed, that is, the molecules of interest should be “desolvated.” Desolvation can, for example, be achieved by interacting the droplets and solvated ions with a strong countercurrent flow (6-9 l/m) of a heated gas before the ions enter into the vacuum of the mass analyzer.

Other well-known ionization methods may also be used. For example, electron ionization (also known as electron bombardment and electron impact), atmospheric pressure chemical ionization (APCI), fast atom Bombardment (FAB), or chemical ionization (CI).

Immediately following ionization, gas phase ions enter a region of the mass spectrometer known as the mass analyzer. The mass analyzer is used to separate ions within a selected range of mass to charge ratios. This is an important part of the instrument because it plays a large role in the instrument's accuracy and mass range. Ions are typically separated by magnetic fields, electric fields, and/or measurement of the time an ion takes to travel a fixed distance.

If all ions with the same charge enter a magnetic field with identical kinetic energies a definite velocity will be associated with each mass and the radius will depend on the mass. Thus a magnetic field can be used to separate a monoenergetic ion beam into its various mass components. Magnetic fields will also cause ions to form fragment ions. If there is no kinetic energy of separation of the fragments the two fragments will continue along the direction of motion with unchanged velocity. Generally, some kinetic energy is lost during the fragmentation process creating non-integer mass peak signals which can be easily identified. Thus, the action of the magnetic field on fragmented ions can be used to give information on the individual fragmentation processes taking place in the mass spectrometer.

Electrostatic fields exert radial forces on ions attracting them towards a common center. The radius of an ion's trajectory will be proportional to the ion's kinetic energy as it travels through the electrostatic field. Thus an electric field can be used to separate ions by selecting for ions that travel within a specific range of radii which is based on the kinetic energy and is also proportion to the mass of each ion.

Quadrupole mass analyzers have been used in conjunction with electron ionization sources since the 1950s. Quadrupoles are four precisely parallel rods with a direct current (DC) voltage and a superimposed radio-frequency (RF) potential. The field on the quadrupoles determines which ions are allowed to reach the detector. The quadrupoles thus function as a mass filter. As the field is imposed, ions moving into this field region will oscillate depending on their mass-to-charge ratio and, depending on the radio frequency field, only ions of a particular m/z can pass through the filter. The m/z of an ion is therefore determined by correlating the field applied to the quadrupoles with the ion reaching the detector. A mass spectrum can be obtained by scanning the RF field. Only ions of a particular m/z are allowed to pass through.

Electron ionization coupled with quadrupole mass analyzers can be employed in practicing the instant invention. Quadrupole mass analyzers have found new utility in their capacity to interface with electrospray ionization. This interface has three primary advantages. First, quadrupoles are tolerant of relatively poor vacuums (˜5×10⁻⁵ torr), which makes it well-suited to electrospray ionization since the ions are produced under atmospheric pressure conditions. Secondly, quadrupoles are now capable of routinely analyzing up to an m/z of 3000, which is useful because electrospray ionization of proteins and other biomolecules commonly produces a charge distribution below m/z 3000. Finally, the relatively low cost of quadrupole mass spectrometers makes them attractive as electrospray analyzers.

The ion trap mass analyzer was conceived of at the same time as the quadrupole mass analyzer. The physics behind both of these analyzers is very similar. In an ion trap the ions are trapped in a radio frequency quadrupole field. One method of using an ion trap for mass spectrometry is to generate ions externally with ESI or MALDI, using ion optics for sample injection into the trapping volume. The quadrupole ion trap typically consist of a ring electrode and two hyperbolic endcap electrodes. The motion of the ions trapped by the electric field resulting from the application of RF and DC voltages allows ions to be trapped or ejected from the ion trap. In the normal mode the RF is scanned to higher voltages, the trapped ions with the lowest m/z and are ejected through small holes in the endcap to a detector (a mass spectrum is obtained by resonantly exciting the ions and thereby ejecting from the trap and detecting them). As the RF is scanned further, higher m/z ratios become are ejected and detected. It is also possible to isolate one ion species by ejecting all others from the trap. The isolated ions can subsequently be fragmented by collisional activation and the fragments detected. The primary advantages of quadrupole ion traps is that multiple collision-induced dissociation experiments can be performed without having multiple analyzers. Other important advantages include its compact size, and the ability to trap and accumulate ions to increase the signal-to-noise ratio of a measurement.

Quadrupole ion traps can be used in conjunction with electrospray ionization MS/MS experiments in the instant invention.

The earliest mass analyzers separated ions with a magnetic field. In magnetic analysis, the ions are accelerated (using an electric field) and are passed into a magnetic field. A charged particle traveling at high speed passing through a magnetic field will experience a force, and travel in a circular motion with a radius depending upon the m/z and speed of the ion. A magnetic analyzer separates ions according to their radii of curvature, and therefore only ions of a given m/z will be able to reach a point detector at any given magnetic field. A primary limitation of typical magnetic analyzers is their relatively low resolution.

In order to improve resolution, single-sector magnetic instruments have been replaced with double-sector instruments by combining the magnetic mass analyzer with an electrostatic analyzer. The electric sector acts as a kinetic energy filter allowing only ions of a particular kinetic energy to pass through its field, irrespective of their mass-to-charge ratio. Given a radius of curvature, R, and a field, E, applied between two curved plates, the equation R=2V/E allows one to determine that only ions of energy V will be allowed to pass. Thus, the addition of an electric sector allows only ions of uniform kinetic energy to reach the detector, thereby increasing the resolution of the two sector instrument to 100,000. Magnetic double-focusing instrumentation is commonly used with FAB and El ionization, however they are not widely used for electrospray and MALDI ionization sources primarily because of the much higher cost of these instruments. But in theory, they can be employed to practice the instant invention.

ESI and MALDI-MS commonly use quadrupole and time-of-flight mass analyzers, respectively. The limited resolution offered by time-of-flight mass analyzers, combined with adduct formation observed with MALDI-MS, results in accuracy on the order of 0.1% to a high of 0.01%, while ESI typically has an accuracy on the order of 0.01%. Both ESI and MALDI are now being coupled to higher resolution mass analyzers such as the ultrahigh resolution (>10⁵) mass analyzer. The result of increasing the resolving power of ESI and MALDI mass spectrometers is an increase in accuracy for biopolymer analysis.

Fourier-transform ion cyclotron resonance (FTMS) offers two distinct advantages, high resolution and the ability to tandem mass spectrometry experiments. FTMS is based on the principle of a charged particle orbiting in the presence of a magnetic field. While the ions are orbiting, a radio frequency (RF) signal is used to excite them and as a result of this RF excitation, the ions produce a detectable image current. The time-dependent image current can then be Fourier transformed to obtain the component frequencies of the different ions which correspond to their m/z.

Coupled to ESI and MALDI, FTMS offers high accuracy with errors as low as ±0.001%. The ability to distinguish individual isotopes of a protein of mass 29,000 is demonstrated.

A time-of-flight (TOF) analyzer is one of the simplest mass analyzing devices and is commonly used with MALDI ionization. Time-of-flight analysis is based on accelerating a set of ions to a detector with the same amount of energy. Because the ions have the same energy, yet a different mass, the ions reach the detector at different times. The smaller ions reach the detector first because of their greater velocity and the larger ions take longer, thus the analyzer is called time-of-flight because the mass is determine from the ions' time of arrival.

The arrival time of an ion at the detector is dependent upon the mass, charge, and kinetic energy of the ion. Since kinetic energy (KE) is equal to ½ mv² or velocity v=(2KE/m)^{1/2}, ions will travel a given distance, d, within a time, t, where t is dependent upon their m/z.

The magnetic double-focusing mass analyzer has two distinct parts, a magnetic sector and an electrostatic sector. The magnet serves to separate ions according to their mass-to-charge ratio since a moving charge passing through a magnetic field will experience a force, and travel in a circular motion with a radius of curvature depending upon the m/z of the ion. A magnetic analyzer separates ions according to their radii of curvature, and therefore only ions of a given m/z will be able to reach a point detector at any given magnetic field. A primary limitation of typical magnetic analyzers is their relatively low resolution. The electric sector acts as a kinetic energy filter allowing only ions of a particular kinetic energy to pass through its field, irrespective of their mass-to-charge ratio. Given a radius of curvature, R, and a field, E, applied between two curved plates, the equation R=2V/E allows one to determine that only ions of energy V will be allowed to pass. Thus, the addition of an electric sector allows only ions of uniform kinetic energy to reach the detector, thereby increasing the resolution of the two sector instrument.

The new ionization techniques are relatively gentle and do not produce a significant amount of fragment ions, this is in contrast to electron ionization (El) which produces many fragment ions. To generate more information on the molecular ions generated in the ESI and MALDI ionization sources, it has been necessary to apply techniques such as tandem mass spectrometry (MS/MS), to induce fragmentation. Tandem mass spectrometry (abbreviated MSn—where n refers to the number of generations of fragment ions being analyzed) allows one to induce fragmentation and mass analyze the fragment ions. This is accomplished by collisionally generating fragments from a particular ion and then mass analyzing the fragment ions.

Tandem mass spectrometry or post source decay is used for proteins that cannot be identified by peptide-mass matching or to confirm the identity of proteins that are tentatively identified by an error-tolerant peptide mass search, described above. This method combines two consecutive stages of mass analysis to detect secondary fragment ions that are formed from a particular precursor ion. The first stage serves to isolate a particular ion of a particular peptide (polypeptide) of interest based on its m/z. The second stage is used to analyze the product ions formed by spontaneous or induced fragmentation of the selected ion precursor. Interpretation of the resulting spectrum provides limited sequence information for the peptide of interest. However, it is faster to use the masses of the observed peptide fragment ions to search an appropriate protein sequence database and identify the protein as described in Griffin et al, Rapid Commun. Mass. Spectrom. 1995, 9: 1546. Peptide fragment ions are produced primarily by breakage of the amide bonds that join adjacent amino acids. The fragmentation of peptides in mass spectrometry has been well described (Falick et al., J. Am Soc. Mass Spectrom. 1993, 4, 882-893; Bieniann, K., Biomed. Environ. Mass Spectrom. 1988, 16, 99-111).

For example, fragmentation can be achieved by inducing ion/molecule collisions by a process known as collision-induced dissociation (CID) or also known as collision-activated dissociation (CAD). CID is accomplished by selecting an ion of interest with a mass filter/analyzer and introducing that ion into a collision cell. A collision gas (typically Ar, although other noble gases can also be used) is introduced into the collision cell, where the selected ion collides with the argon atoms, resulting in fragmentation. The fragments can then be analyzed to obtain a fragment ion spectrum. The abbreviation MSn is applied to processes which analyze beyond the initial fragment ions (MS2) to second (MS3) and third generation fragment ions (MS4). Tandem mass analysis is primarily used to obtain structural information, such as protein or polypeptide sequence, in the instant invention.

In certain instruments, such as those by JEOL USA, Inc. (Peabody, Mass.), the magnetic and electric sectors in any JEOL magnetic sector mass spectrometer can be scanned together in “linked scans” that provide powerful MS/MS capabilities without requiring additional mass analyzers. Linked scans can be used to obtain product-ion mass spectra, precursor-ion mass spectra, and constant neutral-loss mass spectra. These can provide structural information and selectivity even in the presence of chemical interferences. Constant neutral loss spectrum essentially “lifts out” only the interested peaks away from all the background peaks, hence removing the need for class separation and purification. Neutral loss spectrum can be routinely generated by a number of commercial mass spectrometer instruments (such as the one used in the Example section). JEOL mass spectrometers can also perform fast linked scans for GC/MS/MS and LC/MS/MS experiments.

Once the ion passes through the mass analyzer it is then detected by the ion detector, the final element of the mass spectrometer. The detector allows a mass spectrometer to generate a signal (current) from incident ions, by generating secondary electrons, which are further amplified. Alternatively some detectors operate by inducing a current generated by a moving charge. Among the detectors described, the electron multiplier and scintillation counter are probably the most commonly used and convert the kinetic energy of incident ions into a cascade of secondary electrons. Ion detection can typically employ Faraday Cup, Electron Multiplier, Photomultiplier Conversion Dynode (Scintillation Counting or Daly Detector), High-Energy Dynode Detector (HED), Array Detector, or Charge (or Inductive) Detector.

The introduction of computers for MS work entirely altered the manner in which mass spectrometry was performed. Once computers were interfaced with mass spectrometers it was possible to rapidly perform and save analyses. The introduction of faster processors and larger storage capacities has helped launch a new era in mass spectrometry. Automation is now possible allowing for thousands of samples to be analyzed in a single day. The use of computer also helps to develop mass spectra databases which can be used to store experimental results. Software packages not only helped to make the mass spectrometer more user friendly but also greatly expanded the instrument's capabilities.

The ability to analyze complex mixtures has made MALDI and ESI very useful for the examination of proteolytic digests, an application otherwise known as protein mass mapping. Through the application of sequence specific proteases, protein mass mapping allows for the identification of protein primary structure. Performing mass analysis on the resulting proteolytic fragments thus yields information on fragment masses with accuracy approaching ±5 ppm, or ±0.005 Da for a 1,000 Da peptide. The protease fragmentation pattern is then compared with the patterns predicted for all proteins within a database and matches are statistically evaluated. Since the occurrence of Arg and Lys residues in proteins is statistically high, trypsin cleavage (specific for Arg and Lys) generally produces a large number of fragments which in turn offer a reasonable probability for unambiguously identifying the target protein.

The primary tools in these protein identification experiments are mass spectrometry, proteases, and computer-facilitated data analysis. As a result of generating intact ions, the molecular weight information on the peptides/proteins are quite unambiguous. Sequence specific enzymes can then provide protein fragments that can be associated with proteins within a database by correlating observed and predicted fragment masses. The success of this strategy, however, relies on the existence of the protein sequence within the database. With the availability of the human genome sequence (which indirectly contain the sequence information of all the proteins in the human body) and genome sequences of other organisms (mouse, rat, Drosophila, C. elegans, bacteria, yeasts, etc.), identification of the proteins can be quickly determined simply by measuring the mass of proteolytic fragments.

Representative mass spectrometry instruments useful for practicing the instant invention are described in detail in the Examples. A skilled artisan should readily understand that other similar instruments with equivalent function/specification, either commercially available or user modified, are suitable for practicing the instant invention.

Protease Digestion

Prior to analysis by mass spectrometry, the protein may be chemically or enzymatically digested. For protein bands from gels, the protein sample in the gel slice may be subjected to in-gel digestion. (see Shevchenko A. et al., Mass Spectrometric Sequencing of Proteins from Silver Stained Polyacrylamide Gels. Analytical Chemistry 1996, 58: 850).

One aspect of the instant invention is that peptide fragments ending with lysine or arginine residues can be used for sequencing with tandem mass spectrometry. While trypsin is the preferred the protease, many different enzymes can be used to perform the digestion to generate peptide fragments ending with Lys or Arg residues. For instance, in page 886 of a 1979 publication of Enzymes (Dixon, M. et al. ed., 3rd edition, Academic Press, New York and San Francisco, the content of which is incorporated herein by reference), a host of enzymes are listed which all have preferential cleavage sites of either Arg- or Lys- or both, including Trypsin [EC 3.4.21.4], Thrombin [EC 3.4.21.5], Plasmin [EC 3.4.21.7], Kallikrein [EC 3.4.21.8], Acrosin [EC 3.4.21.10], and Coagulation factor Xa [EC 3.4.21.6]. Particularly, Acrosin is the Trypsin-like enzyme of spermatoza, and it is not inhibited by α1-antitrypsin. Plasmin is cited to have higher selectivity than Trypsin, while Thrombin is said to be even more selective. However, this list of enzymes are for illustration purpose only and is not intended to be limiting in any way. Other enzymes known to reliably and predictably perform digestions to generate the polypeptide fragments as described in the instant invention are also within the scope of the invention.

BLAST Search

The raw data of mass spectrometry will be compared to public, private or commercial databases to determine the identity of polypeptides.

BLAST search can be performed at the NCBI's (National Center for Biotechnology Information) BLAST website. According to the NCBI BLAST website, BLAST® (Basic Local Alignment Search Tool) is a set of similarity search programs designed to explore all of the available sequence databases regardless of whether the query is protein or DNA. The BLAST programs have been designed for speed, with a minimal sacrifice of sensitivity to distant sequence relationships. The scores assigned in a BLAST search have a well-defined statistical interpretation, making real matches easier to distinguish from random background hits. BLAST uses a heuristic algorithm which seeks local as opposed to global aligmnents and is therefore able to detect relationships among sequences which share only isolated regions of similarity (Altschul et al., 1990, J. Mol. Biol. 215: 403-10). The BLAST website also offer a “BLAST course,” which explains the basics of the BLAST algorithm, for a better understanding of BLAST.

For protein sequence search, several protein-protein BLAST can be used. Protein BLAST allows one to input protein sequences and compare these against other protein sequences.

“Standard protein-protein BLAST” takes protein sequences in FASTA format, GenBank Accession numbers or GI numbers and compares them against the NCBI protein databases (see below).

“PSI-BLAST” (Position Specific Iterated BLAST) uses an iterative search in which sequences found in one round of searching are used to build a score model for the next round of searching. Highly conserved positions receive high scores and weakly conserved positions receive scores near zero. The profile is used to perform a second (etc.) BLAST search and the results of each “iteration” used to refine the profile. This iterative searching strategy results in increased sensitivity.

“PHI-BLAST” (Pattern Hit Initiated BLAST) combines matching of regular expression pattern with a Position Specific iterative protein search. PHI-BLAST can locate other protein sequences which both contain the regular expression pattern and are homologous to a query protein sequence.

“Search for short, nearly exact sequences” is an option similar to the standard protein-protein BLAST with the parameters set automatically to optimize for searching with short sequences. A short query is more likely to occur by chance in the database. Therefore increasing the Expect value threshold, and also lowering the word size is often necessary before results can be returned. Low Complexity filtering has also been removed since this filters out larger percentage of a short sequence, resulting in little or no query sequence remaining. Also for short protein sequence searches the Matrix is changed to PAM-30 which is better suited to finding short regions of high similarity.

The databases that can be searched by the BLAST program is user selected, and is subject to frequent updates at NCBI. The most commonly used ones are:

Nr: All non-redundant GenBank CDS translations+PDB+SwissProt+PIR+PRF;

Month: All new or revised GenBank CDS translation+PDB+SwissProt+PIR+PRF released in the last 30 days;

Swissprot: Last major release of the SWISS-PROT protein sequence database (no updates);

Drosophila genome: Drosophila genome proteins provided by Celera and Berkeley Drosophila Genome Project (BDGP);

S. cerevisiae: Yeast (Saccharomyces cerevisiae) genomic CDS translations;

Ecoli: Escherichia coli genomic CDS translations;

Pdb: Sequences derived from the 3-dimensional structure from Brookhaven Protein Data Bank;

Alu: Translations of select Alu repeats from REPBASE, suitable for masking Alu repeats from query sequences. It is available by anonymous FTP from the NCBI website. See “Alu alert” by Claverie and Makalowski, Nature vol. 371, page 752 (1994).

Some of the BLAST databases, like SwissProt, PDB and Kabat are complied outside of NCBI. Other like ecoli, dbEST and month, are subsets of the NCBI databases. Other “virtual Databases” can be created using the “Limit by Entrez Query” option.

The Welcome Trust Sanger Institute offer the Ensembl software system which produces and maintains automatic annotation on eukaryotic genomes. All data and codes can be downloaded without constraints from the Sanger Centre website. The Centre also provides the Ensembl's International Protein Index databases which contain more than 90% of all known human protein sequences and additional prediction of about 10,000 proteins with supporting evidence. All these can be used for database search purposes.

In addition, many commercial databases are also available for search purposes. For example, Celera has sequenced the whole human genome and offers commercial access to its proprietary annotated sequence database (Discovery™ database).

Various software programs can be employed to search these databases. The probability search software Mascot (Matrix Science Ltd.). Mascot utilizes the Mowse search algorithm and scores the hits using a probabilistic measure (Perkins et al., 1999, Electrophoresis 20: 355 13567, the entire contents are incorporated herein by reference). The Mascot score is a function of the database utilized, and the score can be used to assess the null hypothesis that a particular match occurred by chance. Specifically, a Mascot score of 46 implies that the chance of a random hit is less than 5%. However, the total score consists of the individual peptide scores, and occasionally, a high total score can derive from many poor hits. To exclude this possibility, only “high quality” hits—those with a total score>46 with at least a single peptide match with a score of 30 ranking number 1—are considered.

Other similar softwares can also be used according to manufacturer's suggestion.

PubMed, available via the NCBI Entrez retrieval system, was developed by the National Center for Biotechnology Information (NCBI) at the National Library of Medicine (NLM), located at the National Institutes of Health (NIH). The PubMed database was developed in conjunction with publishers of biomedical literature as a search tool for accessing literature citations and linking to full-text journal articles at web sites of participating publishers.

Publishers participating in PubMed electronically supply NLM with their citations prior to or at the time of publication. If the publisher has a web site that offers full-text of its journals, PubMed provides links to that site, as well as sites to other biological data, sequence centers, etc. User registration, a subscription fee, or some other type of fee may be required to access the full-text of articles in some journals.

In addition, PubMed provides a Batch Citation Matcher, which allows publishers (or other outside users) to match their citations to PubMed entries, using bibliographic information such as journal, volume, issue, page number, and year. This permits publishers easily to link from references in their published articles directly to entries in PubMed.

PubMed provides access to bibliographic information which includes MEDLINE as well as:

The out-of-scope citations (e.g., articles on plate tectonics or astrophysics) from certain MEDLINE journals, primarily general science and chemistry journals, for which the life sciences articles are indexed for MEDLINE.

Citations that precede the date that a journal was selected for MEDLINE indexing.

Some additional life science journals that submit full text to PubMed Central and receive a qualitative review by NLM.

PubMed also provides access and links to the integrated molecular biology databases included in NCBI's Entrez retrieval system. These databases contain DNA and protein sequences, 3-D protein structure data, population study data sets, and assemblies of complete genomes in an integrated system.

MEDLINE is the NLM's premier bibliographic database covering the fields of medicine, nursing, dentistry, veterinary medicine, the health care system, and the pre-clinical sciences. MEDLINE contains bibliographic citations and author abstracts from more than 4,300 biomedical journals published in the United States and 70 other countries. The file contains over 11 million citations dating back to the mid-1960's. Coverage is worldwide, but most records are from English-language sources or have English abstracts.

PubMed's in-process records provide basic citation information and abstracts before the citations are indexed with NLM's MeSH Terms and added to MEDLINE. New in process records are added to PubMed daily and display with the tag [PubMed—in process]. After MeSH terms, publication types, GenBank accession numbers, and other indexing data are added, the completed MEDLINE citations are added weekly to PubMed.

Citations received electronically from publishers appear in PubMed with the tag [PubMed—as supplied by publisher]. These citations are added to PubMed Tuesday through Saturday. Most of these progress to In Process, and later to MEDLINE status. Not all citations will be indexed for MEDLINE and are tagged, [PubMed—as supplied by publisher].

The Batch Citation Matcher allows users to match their own list of citations to PubMed entries, using bibliographic information such as journal, volume, issue, page number, and year. The Citation Matcher reports the corresponding PMID. This number can then be used to easily to link to PubMed. This service is frequently used by publishers or other database providers who wish to link from bibliographic references on their web sites directly to entries in PubMed.

As used herein, nr database includes all non-redundant GenBank CDS translations+PDB+SwissProt+PIR+PRF according to the BLAST website.

The E-value for an alignment score “S” represents the number of hits with a score equal to or better than “S” that would be “expected” by chance (the background noise) when searching a database of a particular size. In BLAST 2.0, the E-value is used instead of a P-value (probability) to report the significance of a match. The default E-value for blastn, blastp, blastx and tblastn is 10. At this setting, 10 hits with scores equal to or better than the defined alignment score, S, are expected to occur by chance (in a search of the database using a random query with similar length). The E-value can be increased or decreased to alter the stringency of the search. Increase the E-value to 1000 or more when searching with a short query, since it is likely to be found many times by chance in a given database. Other information regarding the BLAST program can be found at the NCBI BLAST website.

The invention also uses standard laboratory techniques, including but are not limited to recombination-based molecular cloning, yeast cell culture, immunoprecipitation, SDS-PAGE electrophoresis, protein complex isolation, in-gel protease digestion, etc. Such information can be readily found in a number of standard laboratory manuals such as Current Protocols in Cell Biology (CD-ROM Edition, ed. by Juan S. Bonifacino, Jennifer Lippincott-Schwartz, Joe B. Harford, and Kenneth M. Yamada, John Wiley & Sons, 1999).

### EXAMPLE

### Phosphoproteome Analysis by Mass Spectrometry

Following the methodology of the present invention, it is now possible to characterize most, if not all, phosphoproteins from a whole cell lysate in a single experiment. Proteins were digested with trypsin and the resulting peptides are then converted to methyl esters, enriched for phosphopetpides by immobilized metal affinity chromatography (IMAC), and analyzed by nanoflow HPLC/electrospray ionization mass spectrometry.

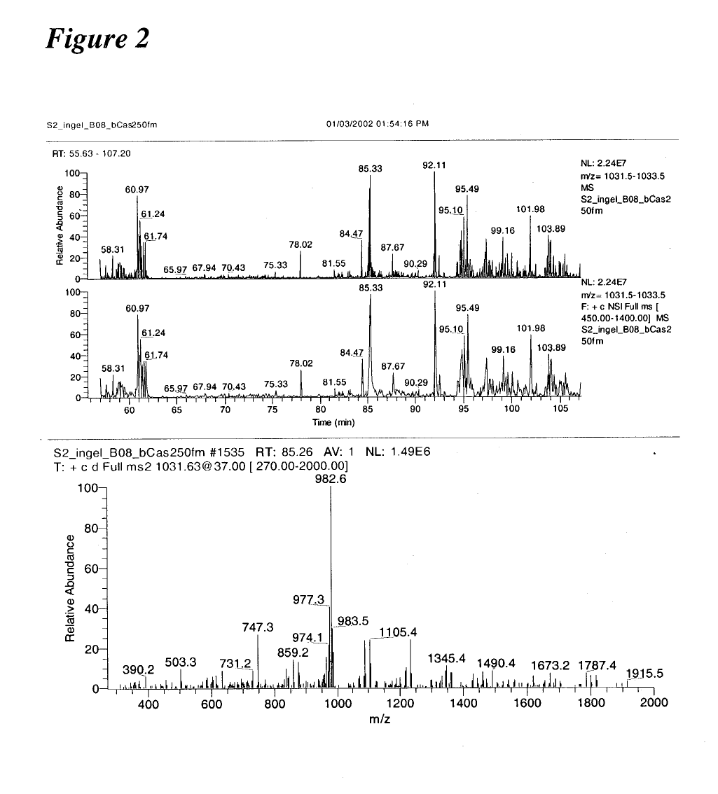

Initial experiments were conducted using a prototype version of the invention. In one such experiment, B-casein was digested with trypsin and analyzed using the invention. Results of these experiments are shown in FIG. 2.

More than a 1,000 phosphopeptides were detected when the methodology was applied to the analysis of a whole cell lysate from S. cerevisiae. Sequences, including 383 sites of phosphorylation derived from 216 peptides were determined. Of these 60 were singly phosphorylated, 145 doubly phosphorylated, and 11 triply phosphorylated. To validate the approach, the results were compared with the literature, revealing 18 previously identified sites, including the doubly phosphorylated motif pTXpY derived from the activation loop of two MAP kinases. We note that the methodology can easily be extended to display and quantitate differential expression of phosphoproteins in two different cell systems, and therefore demonstrates an approach for “phosphoprofiling” as a measure of cellular states.

We prepared a standard mixture of tryptic peptides containing a single phosphopeptide and then analyzed the mixture before and after converting the peptides to the corresponding methyl esters. This rendered the IMAC selective for phosphopeptides, and eliminated confounding binding through carboxylate groups. Equimolar quantities of glyceraldehyde 3 phosphate dehydrogenase, bovine serum albumin, carbonic anydrase, ubiquitin, and β-lactoglobulin were digested with trypsin (approximately 125 predicted cleavage sites) and then combined with the phosphopeptide DRVpYIHPF (SEQ ID No: 1) (lower case p precedes a phosphorylated residue), to give a mixture that contained tryptic peptides at the 2 pmol/μl level and phosphopeptide at the 10 fmol/μl level. All experiments were performed on 0.5 μl aliquots of this solution.

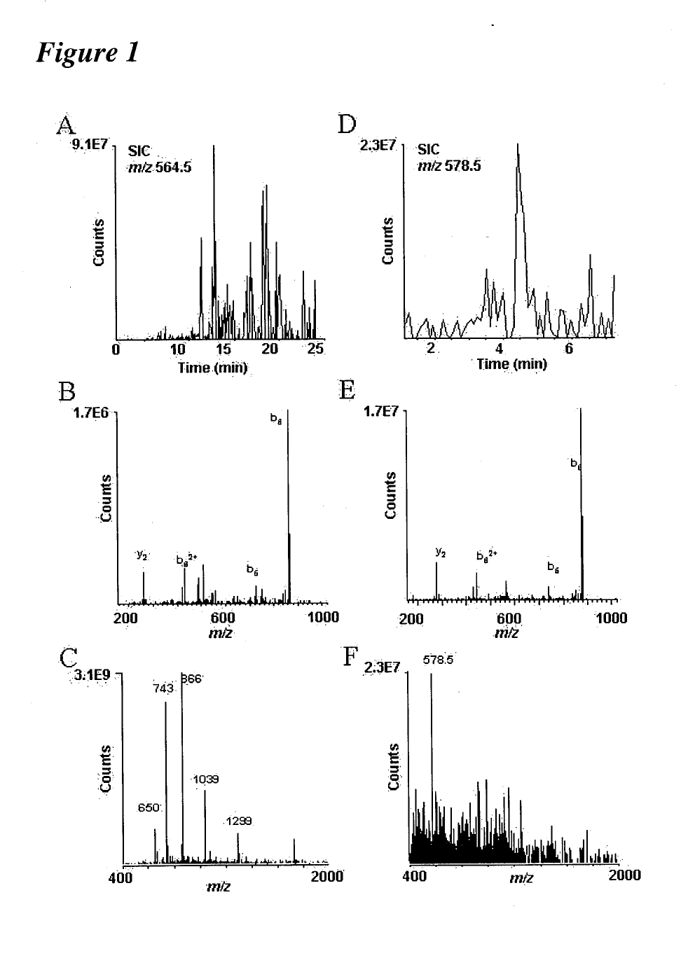

Shown in FIG. 1 are the results obtained when a 0.5 μl aliquot of the standard mixture was analyzed by a combination of IMAC^{5,6} and nanoflow-HPLC on the LCQ ion trap mass spectrometer. In this experiment, the instrument was set to cycle between two different scan functions every 2 sec throughout the HPLC gradient. Electrospray ionization spectra were recorded in the first of the two scans. MS/MS spectra on the (M+2H)⁺⁺ ion of the phosphopeptide, DRVpYIHPF (SEQ ID No: 1, m/z 564.5) were recorded in the second scan of the cycle. FIG. 1A shows a selected-ion-chromatogram (SIC) or plot of the ion current observed for m/z 564.5 as a function of scan number. Note that a signal at this m/z value is observed at numerous points in the chromatogram. Only ions at m/z 564.5 in scans 610-616 fragment to generate MS/MS spectra characteristic of the phosphopeptide, DRVpYIHPF (SEQ ID No: 1, FIG. 1B). We conclude that DRVpYIHPF (SEQ ID No: 1) elutes from the HPLC column in scans 610-616.

Displayed in FIG. 1C is an electrospray ionization mass spectrum recorded during this same time period. Note that the spectrum contains signals of high intensity (ion currents of 1-3×10⁹) corresponding to nonphosphorylated tryptic peptides in the mixture but no signal above the chemical noise level for the phosphopeptide (m/z 564.5). We conclude that tryptic peptides containing multiple carboxylic acid groups can bind efficiently to the IMAC column, elute during the HPLC gradient, and suppress the signal from trace level phosphopeptides in the mixture.

To prevent binding of non-phosphorylated peptides to the IMAC column, all peptides in the standard mixture were converted to the corresponding peptide methyl esters and a 0.5 μl aliquot was then analyzed by the protocol outlined above. To detect the phosphopeptide in which both carboxylic acid groups had been esterified, MS/MS spectra were recorded on the (M+2H)⁺⁺ ion at m/z 578.5. The SIC for m/z 578.5 (FIG. 1D) suggests that the phosphopeptide dimethyl ester elutes during scans 151-163. Indeed, MS/MS spectra (FIG. 1E) recorded in this time window all contain the predicted fragments expected for the dimethyl ester of DRVpYIHPF (SEQ ID No: 1). FIG. 1F shows an electrospray ionization mass spectrum recorded in the same area of the chromatogram (scan #154). Note that the parent ion, m/z 578.5 for the phosphopeptide dimethyl ester is now observed with a signal/noise of 3/1 and an ion current of 2×10⁷. This signal level on the LCQ is not atypical for phosphopeptpide samples at the 3-5 fmol level. Note also that signals above the chemical noise (ion current of 1×10⁷) for nonphosphorylated tryptic peptides no longer appear in this electrospray ionization spectrum or in any other spectrum recorded throughout the entire chromatogram. We conclude that conversion of carboxylic acid groups to methyl esters reduces nonspecific binding by at least two orders of magnitude and allows detection of phosphopeptides in complex mixtures down to the level of at least 5 fmol with the LCQ instrument.

To further evaluate the above protocol, we next analyzed a protein pellet (500 μg) obtained from a whole cell lysate of S. cerevisiae. If the average mol. wt. of yeast proteins is 25 kDa and half the genome is expressed and isolated in the pellet, then the average quantity each protein in the sample is expected to be approximately 5 pmol. If one makes the further assumption that 30% of expressed proteins contain at least one covalently bound phosphate, the total number of phosphoproteins in the sample could easily exceed 1,000. To evaluate this possibility, the pellet was digested with trypsin and the resulting peptides were converted to peptide methyl esters. One fifth of the resulting mixture was then fractionated by IMAC and analyzed by nano-flow HPLC on the LCQ ion trap mass spectrometer. Spectra were acquired with the instrument operating in the data-dependent mode throughout the HPLC gradient. Every 12-15 sec, the instrument cycled through acquisition of a full scan mass spectrum and 5 MS/MS spectra recorded sequentially on the 5 most abundant ions present in the initial MS scan. More than 1,500 MS/MS spectra were recorded in this mode of operation during the chromatographic separation.

Data acquired in the above experiment were analyzed both by a computer algorithm, the Neutral Loss Tool, and also by SEQUEST. The Neutral Loss Tool searches MS/MS spectra for fragment ions formed by loss of phosphoric acid, 32.6, 49 or 98 Da from the (M+3H)⁺⁺⁺, (M+2H)⁺⁺ and (M+H)⁺ ions, respectively. Phosphoserine and phosphothreonine, but not phosphotyrosine, lose phosphoric acid readily during the collision activation dissociation process in the ion trap mass spectrometer. Thus, appearance of fragment ions 32.6, 49 or 98 Da below the triply, doubly or singly charged precursor ions in peptide MS/MS spectra strongly suggests that the peptide contains at least one phosphoserine or phosphothreonine residue. In the above experiment, more than 1,000 different phosphoserine or phosphothreonine containing peptides were detected in the yeast whole cell lysate with the Neutral Loss Tool.

To identify phosphopeptides in the above sample, MS/MS spectra were searched with the SEQUEST algorithm against yeast protein database (obtained from the Saccharomyces Genome Database (SGD) http://genome-www.stanford.edu/Saccharomyces/). Of the 216 sequence confirmed, 60 (28%) are singly phosphorylated, 145 (67%) are doubly phosphorylated, and 11 (5%) are triply phosphorylated.

This clearly indicates the potential of the phosphoprofiling approach as a measure of cellular activation states. In fact, we identified 171 different proteins, including abundant species such as the heat shock proteins and those involved in carbohydrate metabolism, and protein synthesis. Rare proteins such as the cell cycle regulatory molecules and cytoplasmic proteins, were also observed. Of the 216 confirmed peptide sequences, 66 have sequences that correspond to a codon bias of less than 0.1 and are therefore likely to be expressed in low copy number.

Eighty-five additional phosphopeptides were identified by recording MS/MS on the sample eluted from the IMAC column after it had been treated with alkaline phosphatase to remove covalently bound phosphate. In this experiment, peptide methyl esters were eluted from the IMAC column directly to a second column packed with F7m Polyvinyl spheres containing immobilized alkaline phosphatase. Dephosphorylated peptides were then eluted to the standard nano-flow HPLC column and analyzed on the LCQ using the data dependent scan protocol described above. This approach has the advantage that the resulting MS/MS spectra usually contain a larger number of abundant, sequence-dependent, fragment ions than those recorded on the corresponding phosphorylated analogs. This, in turn, improves the likelihood that the SEQUEST algorithm will find a unique match in the protein database. The disadvantage of the protocol is that the resulting MS/MS spectra no longer contain information on the number and location of the phosphorylated residues within the peptide.

Also, we note that the above methodology can be modified easily to allow quantitation and/or differential display of phosphoproteins expressed in two different samples. For this experiment, peptides are converted to methyl esters from one sample with do-methanol and from the other sample with d₃-methanol. The two samples are combined, fractionated by IMAC, and the resulting mixture of labeled and unlabeled phosphopeptides is then analyzed by nanoflow HPLC/electrospray ionization on a newly constructed Fourier transform mass spectrometer. This instrument operates with a detection limit in the low attomole level. Signals for peptides present in both samples appear as doublets separated by n(3Da)/z ( where n=the number of carboxylic acid groups in the peptide and z=the charge on the peptide). The ratio of the two signals in the doublet changes as a function of expression level of the particular phosphoprotein in each sample. Peptides of interest are then targeted for sequence analysis in a subsequent analysis performed on the ion trap instrument as discussed above.

Fractionation of peptides on these columns is based on their affinity for Fe+³ that is coordinated to chelating agents covalently attached to the packing material⁷.

Protein extraction from S. cerevisiae. Yeast strain 2124 MATa ade2-1, ade6-1, leu2-3,112, ura3-52, his3Δ1, trp1-289, can1 cyh2 bar1::KAN (40 ml) was grown in YPD at 23° C. to a density of 1×10⁷ cells/ml. The cell pellet was resuspended in 1.5 ml of Trizol (Gibco-BRL) and cell lysis was performed by homogenization with glass beads in 3 consecutive sessions of 45 sec each in a Fastprep FP 120 shaker (Savant). Total yeast protein, free of nucleic acids, was extracted from this yeast lysate using Trizol according to the manufacturer's directions (GibcoBRL). The protein pellet was resuspended in 1% SDS, and dialyzed against 1% SDS using a Slyde-A-Lyzer, 10,000 MW cutoff (Pierce) to remove small molecules and stored at −80° C. To follow the removal of nucleotide, 0.1 μl of a P³² CTP (Amersham-Pharmacia) was added to a 10 ml equivalent of lysed cells. Aliquots were removed after each step in purification and the amount of nucleotide was quantitated by Scintillation with Scintisafe EconoF (Fischer). Yeast protein, 500 μg (approximately 10 nmol), in 500 μl of 100 mM ammonium acetate (pH 8.9), was digested with trypsin (20 μg)(Promega) overnight at 37° C. Solvent was removed by lyophilization and the residue was reconstituted in 400 μl of 2N methanolic HCl and allowed to stand at room temperature for 2 h. Solvent was lyophilized and the resulting peptide methyl esters were dissolved in 120 μl of a solution containing equal parts of methanol, water and acetonitrile. An aliquot corresponding to 20% of this material (2 nmol of yeast protein) was subjected to chromatography and mass spectrometry as described below.

Chromatography. Construction of immobilized metal affinity chromatography (IMAC) columns has been described previously⁹. Briefly, 360 μm O.D.×100 μm I.D. fused silica (Polymicro Technologies, Phoenix, Ariz.) was packed with 8 cm POROS 20 MC (PerSeptive Biosystems, Framingham, Mass.). Columns were activated with 200 μl 100 mM FeCl₃ (Aldrich, Milwaukee, Wis. and loaded with either 0.5 μl of the above standard mixture or sample corresponding to peptides derived from 100 μg (10 nmol) of protein extract from S. cerevisiae. To remove non-specific binding peptides, the column was washed with a solution containing 100 mM NaCl (Aldrich) in acetonitrile (Mallinkrodt, Paris, Ky.), water, and glacial acetic acid (Aldrich) (25:74:1, v/v/v). For sample analysis by mass spectrometry, the affinity column was connected to a fused silica pre-column (6 cm of 360 μm O.D.×100 μm I.D.) packed with 5-20 μm C18 particles (YMC, Wilmington, N.C.). All column connections were made with 1 cm of 0.012″ I.D.×0.060″ O.D. Teflon tubing (Zeus, Orangeburg, S.C.). Phosphopeptides were eluted to the pre-column with 10 μl 50 mM Na₂HPO₄ (Aldrich) (pH 9.0) and the pre-column was then rinsed with several column volumes of 0.1% acetic acid to remove Na₂HPO₄. The pre-column was connected to the analytical HPLC column (360 μm O.D.×100 μn I.D. fused silica) packed with 6-8 cm of 5 μm C18 particles (YMC, Wilmington, N.C.). One end of this column contained an integrated laser pulled ESI emitter tip (2-4 μm in diameter)¹⁴. Sample elution from the HPLC column to the mass spectrometer was accomplished with a gradient consisting of 0.1% acetic acid and acetonitrile. For removal of phosphate from the tryptic peptides, the IMAC column was connected to a fritted 360 μm O.D.×200 μm I.D. fused silica capillary packed with F7m (Polyvinyl spheres), containing immobilized alkaline phosphatase (MoBiTech, Marco Island, Fla.). Phosphopeptides were eluted from the IMAC column through the phosphatase column onto a precolumn with 25 μL of 1 mM ethylenediaminetetraacetic acid (EDTA) (pH 9.0), and the precolumn was then rinsed with several column volumes of 0.1% acetic acid to remove EDTA. The pre-column was connected to an analytical HPLC column. Sample elution from the HPLC column to the mass spectrometer was accomplished with a gradient consisting of 0.1% acetic acid and acetonitrile.

Mass Spectrometry. All samples were analyzed by nanoflow-HPLC/microelectrospray ionization on a Finnigan LCQ® ion trap (San Jose, Calif.). A gradient consisting of 0-40% B in 60 min, 40-100% B in 5 min (A=100 mM acetic acid in water, B=70% acetonitrile, 100 mM acetic acid in water) flowing at approximately 10 nL/min was used to elute peptides from the reverse-phase column to the mass spectrometer through an integrated electrospray emitter tip¹⁴. Spectra were acquired with the instrument operating in the data-dependent mode throughout the HPLC gradient. Every 12-15 sec, the instrument cycled through acquisition of a full scan mass spectrum and 5 MS/MS spectra (3 Da window; precursor m/z +/−1.5 Da, collision energy set to 40%, dynamic exclusion time of 1 minute) recorded sequentially on the 5 most abundant ions present in the initial MS scan. To perform targeted analysis of the phosphopeptide in the standard mixture, the ion trap mass spectrometer was set to repeat a cycle consisting of a full MS scan followed by an MS/MS scan (collision energy set to 40%) on the (M+2H)⁺⁺ of DRVpYIHPF (SEQ ID No: 1) or its methyl ester (m/z 564.5 and 578.5, respectively). The gradient employed for this experiment was 0-100% B in 30 minutes for the underivatized sample, 0-100% B in 17 minutes for derivatized sample (A=100 mM acetic acid in water, B=70% acetonitrile, 100 mM acetic acid in water).

Database Analysis. All MS/MS spectra recorded on tryptic phosphopeptides derived from the yeast protein extract were searched against the S. cerevisiae protein database by using the SEQUEST algorithm¹⁰. Search parameters included a differential modification of +80 Da (presence or absence of phosphate) on serine, threonine and tyrosine and a static modification of +14 Da (methyl groups) on aspartic acid, glutamic acid, and the C-terminus of each peptide.

References

1. Hubbard, M. J. and Cohen, P. On target with a new mechanism for the regulation of protein phosphorylation. Trends Biochem. Sci. 18, 172-177 (1993).

2. Annan, R., Huddleston, M., Verma, R., Deshaies, R. & Carr, S. A Multidimensional Electrospray MS-Based Approach to Phosphopeptide Mapping. Anal. Chem. 73, 393-404 (2001).

3. Oda, Y., Nagasu, T. & Chait, B. Enrichment analysis of phosphorylated proteins as a tool for probing the phosphoproteome. Nat. Biotechnol. 19, 379-382 (2001).

4. Zhou, H., Watts, J. & Aebersold, R. A systematic approach to the analysis of protein phosphorylation. Nat. Biotechnol. 19, 375-378 (2001).

5. Andersson, L. and Porath, J. Isolation of phosphoproteins by immobilized metal (Fe3+) affinity chromatography. Anal. Biochem. 154, 250-254 (1986b)

6. Michel, H., Hunt, D. F., Shabanowitz, J. and Bennett, J. Tandem mass spectrometry reveals that three photosystem II proteins of spinach chloroplasts contain N-acetyl-O-phosphothreonine at their NH2 termini. J. Biol. Chem. 263, 1123-1130 (1988).

7. Muszynska, G., Dobrowolska, G., Medin, A., Ekman, P. & Porath, J. O. Model studies on iron(III) ion affinity chromatography. II. Interaction of immobilized iron(III) ions with phosphorylated amino acids, peptides and proteins. J. Chrom. 604, 19-28 (1992).

8. Nuwaysir, L. & Stults, J. Electrospray ionization mass spectrometry of phosphopeptides isolated by on-line immobilized metal-ion affinity chromatography. J. Amer. Soc. Mass Spectrom. 4, 662-669 (1993).

9. Zarling, A. L. et al. Phosphorylated peptides are naturally processed and presented by major histocompatibility complex class I molecules in vivo. J. Exp. Med. 192, 1755-1762 (2000).

10. Eng, J., McCormack, A. L. and Yates, J. R. An approach to correlate tandem mass spectral data of peptides with amino acid sequences in a protein database. J. Amer. Soc. Mass Spectrom, 5, 976-989 (1994).

11. Bennetzen, J. L. & Hall, B. D. Codon selection in yeast. J Biol Chem 257, 3026-3031 (1982).

12. Zhang, X. et al. Identification of phosphorylation sites in proteins separated by polyacrylamide gel electrophoresis. Anal Chem 70, 2050-2059 (1998).

13. Amankwa, L. N., Harder, K., Jirik, F. & Aebersold, R. High-sensitivity determination of tyrosine-phosphorylated peptides by on-line enzyme reactor and electrospray ionization mass spectrometry. Prot. Sci. 4, 13-125 (1995).

14. Martin, S. E., Shabanowitz, J., Htnt, D. F. & Marto, J. A. Subfemtomole ms and ms/ms peptide sequence analysis using nano-hplc micro-esi fourier transform ion cyclotron resonance mass spectrometry. Anal Chem 72, 4266-4274 (2000).

We claim:

1. An automated method for identifying modified amino acids within a protein, comprising:
(i) providing one or more protein samples and an affinity capture reagent for isolating, from the protein samples, those proteins which have been post-translationally modified with a moiety of interest;
(ii) processing the protein samples to chemically modify at least one of the C-terminal carboxyl, the N-terminal amine and amino acid side chains of proteins in the protein samples so as to increase the specificity of the affinity capture reagent for those proteins which have been post-translationally modified with the moiety of interest;
(iii) isolating proteins post-translationally with the moiety of interest from the proteins samples using the affinity capture reagent; and,
(iv) determining the identity of isolated proteins by mass spectroscopy;
wherein the method is performed using an apparatus comprising an autosampler, an affinity-capture apparatus and an ion source.

2. The method of claim 1, wherein the proteins are further cleaved into smaller peptide fragments before, after or during the step of processing the protein samples.

3. The method of claim 2, wherein the proteins are fragmented by enzymatic hydrolysis to produce peptide fragments having carboxy-terminal lysine or arginine residues.

4. The method of claim 3, wherein the proteins are fragmented by treatment with trypsin.

5. The method of claim 1, wherein the proteins are mass-modified with isotopic labels before, after or during the step of processing the protein samples.

6. The method of claim 1, wherein the isolated proteins are further separated by reverse phase chromatography before analysis by mass spectroscopy.

7. The method of claim 1, wherein the isolated proteins are identified from analysis using tandem mass spectroscopy techniques.

8. The method of claim 1, wherein the identity of the isolated proteins are determined by searching molecular weight databases for the molecular weight observed by mass spectroscopy for an isolated protein or peptide fragment thereof.

9. The method of claim 1, further comprising including the step of obtaining amino acid sequence mass spectra for the isolated proteins or peptide fragments, and searching one or more sequence databases for the sequence observed for the identified protein or peptide fragment thereof.

10. The method of claim 1, wherein the moiety of interest is a phosphate group.

11. The method of claim 10, wherein the affinity capture reagent is an immobilized metal affinity chromatography medium, and the step of processing the protein samples includes chemically modifying the side chains of glutamic acid and aspartic acid residues to neutral derivatives.

12. The method of claim 11, wherein the side chains of glutamic acid and aspartic acid residues are modified by alkyl-esterification.

13. The method of claim 1, wherein the protein sample is a mixture of different proteins.

14. The method of claim 13, wherein the protein sample is derived from a biological fluid, or a cell or tissue lysates.

15. The method of claim 1, carried out on multiple different protein samples, wherein the proteins or fragments thereof of each protein sample are isotopically labeled in a manner which permits discrimination of mass spectroscopy data between protein samples.

16. A method for analyzing a phosphoproteome, comprising:
(i) for a protein sample, chemically modify the side chains of glutamic acid and aspartic acid residues to neutral derivatives;
(ii) isolating phosphorylated proteins from the protein sample by immobilized metal affinity chromatography;
(iii) determining the identity of isolated proteins by mass spectroscopy.

17. The method of claim 16, the proteins are cleaved into smaller peptide fragments before, after or during the step of chemically modify the glutamic acid and aspartic acid residues.

18. The method of claim 17, wherein the proteins are fragmented by enzymatic hydrolysis to produce peptide fragments having carboxy-terminal lysine or arginine residues.

19. The method of claim 18, wherein the proteins are fragmented by treatment with trypsin.

20. The method of claim 16, wherein the glutamic acid and aspartic acid residues are modified by alkyl-esterification.

21. The method of claim 16, carried out on multiple different protein samples, and proteins which a differentially phosphorylated between two or more protein samples are identified.

22. The method of claim 16, comprising the further step of generating or adding to a database the identity of proteins which are determined to be phosphorylated.

23. A method for identifying a treatment that modulates a modification of amino acid in a target polypeptide, comprising:
(i) providing a protein sample which has been subjected to a treatment of interest;
(ii) using any one of the methods of claims 1-18, determining the identity of proteins which are differentially modified in the treated protein sample relative to an untreated sample or control sample;
(iii) determining whether the treatment results in a pattern of changes in protein modification, relative to the untreated sample or control sample, which meet a preselected criteria.

24. The method of claim 23, wherein the treatment is effected by a compound.

25. The method of claim 24, wherein the compound is a growth factor, a cytokine, a hormone, or a small chemical molecule.

26. The method of claim 23, wherein the compound is from a chemical library.

27. The method of claim 23, wherein the protein sample is isolated from a cell or tissue subjected to the treatment of interest.

28. A method of conducting a drug discovery business, comprising:
(i) by the method of claim 20, determining the identity of a compound that produces a pattern of changes in protein modification, relative to the untreated sample or control sample, which meet a preselected criteria;
(ii) conducting therapeutic profiling of the compound identified in step (i), or further analogs thereof, for efficacy and toxicity in animals; and,
(iii) formulating a pharmaceutical preparation including one or more compounds identified in step (ii) as having an acceptable therapeutic profile.

29. The method of claim 28, including an additional step of establishing a distribution system for distributing the pharmaceutical preparation for sale, and may optionally include establishing a sales group for marketing the pharmaceutical preparation.

30. A method of conducting a drug discovery business, comprising:
(i) by the method of claim 23, determining the identity of a compound that produces a pattern of changes in protein modification, relative to the untreated sample or control sample, which meet a preselected criteria;
(ii) licensing, to a third party, the rights for further drug development of compounds that alter the level of modification of the target polypeptide.

31. A method of conducting a drug discovery business, comprising:
(i) by the method of claim 1, determining the identity of a protein that is post-translationally modified under conditions of interest;
(ii) identify one or more enzymes which catalyze the post-translational modification of the identified protein under the conditions of interest;
(iii) conduct drug screening assays to identify compounds which inhibit or potentiate the enzymes identified in step (ii) and which modulate the post-translational modification of the identified protein under the conditions of interest.
